(19) United States
(12) Patent Application Publication
(10) Pub. No.: US 2022/0331361 A1
(43) Pub. Date: Oct. 20, 2022

(54) GENE TRANSFER VECTORS AND METHODS OF ENGINEERING CELLS

(71) Applicant: CENTURY THERAPEUTICS, INC., Philadelphia, PA (US)

(72) Inventors: Michael Francis NASO, Swarthmore, PA (US); Buddha GURUNG, Chalfont, PA (US); Jill Marinari CARTON, Collegeville, PA (US); John WHEELER, Downingtown, PA (US); Luis Ghira BORGES, Philadelphia, PA (US)

(73) Assignee: CENTURY THERAPEUTICS, INC., Philadelphia, PA (US)

(21) Appl. No.: 17/714,873

(22) Filed: Apr. 6, 2022

Related U.S. Application Data
(60) Provisional application No. 63/171,891, filed on Apr. 7, 2021.

(51) Int. Cl. A61K 35/17; C12N 15/11; C12N 9/22; C12N 15/62; C12N 5/074; C12N 15/85; C12N 15/90; C07K 14/725

(52) U.S. Cl. CPC A61K 35/17; C12N 15/11; C12N 9/22; C12N 15/625; C12N 5/0696; C12N 15/85; C12N 15/907; C07K 14/7051; C12N 2310/20; C12N 2800/80; C12N 2800/107

(57) ABSTRACT

The present disclosure provides compositions and methods for use in genome engineering of induced pluripotent stem cells (iPSCs). Specifically, the methods and compositions described are useful for introducing transgenes into iPSCs such as pluripotent hematopoietic stem cells and/or progenitor cells (HSC/PC) using an CRISPR nuclease-based system (e.g., MAD7 nuclease-based system) and preparing immune-effector cells derived from the iPSCs.

### CROSS-REFERENCE TO RELATED APPLICATIONS

This application claims the benefit of U.S. Provisional Patent Application No. 63/171,891 filed Apr. 7, 2021, which is incorporated by reference herein in its entirety.

### FIELD

The present disclosure is in the field of genome engineering, particularly targeted modification of the genome of a cell.

### REFERENCE TO SEQUENCE LISTING SUBMITTED ELECTRONICALLY

This application contains a sequence listing, which is submitted electronically via EFS-Web as an ASCII formatted sequence listing with a file name “SequenceListing_ST25.txt” and a creation date of Mar. 31, 2022 and having a size of 119 kb. The sequence listing submitted via EFS-Web is part of the specification and is herein incorporated by reference in its entirety.

### BACKGROUND

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. These methods often involve the use of engineered cleavage systems to induce a double strand break (DSB) or a nick in a target DNA sequence such that repair of the break by an error-prone process such as non-homologous end joining (NHEJ) or repair using a repair template (homology directed repair or HDR) can result in the knock-out of a gene or the insertion of a sequence of interest (targeted integration). Cleavage can occur through the use of specific nucleases such as engineered zinc finger nucleases (ZFN), transcription-activator like effector nucleases (TALENs) or CRISPR/Cas systems with an engineered crRNA/tracr RNA (“single guide RNA”) to guide specific cleavage.

Induced pluripotent stem cells, commonly abbreviated as iPS cells or iPSCs, are a type of pluripotent stem cells artificially derived from non-pluripotent cells, typically adult somatic cells, by inserting certain genes. Induced pluripotent stem cells are believed to be identical to natural pluripotent stem cells, such as embryonic stem cells in many respects, for example, in the expression of certain stem cell genes and proteins, chromatin methylation patterns, doubling time, embryoid body formation, teratoma formation, viable chimera formation, and potency and differentiability, but the full extent of the relation to natural pluripotent stem cells is still being assessed. IPS cells were first produced in 2006 (Takahashi et al., 2006) from mouse cells and in 2007 from human cells (Takahashi et al., 2007; Yu et al, 2007). This has been cited as an important advancement in stem cell research, as it has allowed researchers to obtain pluripotent stem cells, which are important in research and potentially have therapeutic uses, without the controversial use of embryos.

Human iPSC technology represents a highly promising and potentially unlimited source of therapeutically viable hematopoietic cells for the treatment of numerous hematological and non-hematological malignancies including cancer. To advance the promise of human iPSC and genomically engineered human iPSC technology as an allogeneic source of hematopoietic cellular therapeutics, it is essential to be able to efficiently and reproducibly generate not only hematopoietic stem and progenitor cells (HSCs) but also immune effector populations, including the diverse subsets of T, B, NKT, and NK lymphoid cells, and progenitor cells thereof having desired genetic modifications. Thus there is a need for methods and complexes for the efficient insertion of genetic elements in human iPSCs for therapeutic use.

### BRIEF SUMMARY

The present disclosure describes compositions and methods for use in genome engineering of cells, such as iPSCs. Specifically, the methods and compositions described relate to compositions and methods for introducing transgenes into iPSCs such as pluripotent hematopoietic stem cells and/or progenitor cells (HSC/PC) and preparing immune-effector cells derived from the iPSCs. More specifically, one aspect of this disclosure relates to a MAD7/gRNA ribonucleoprotein (RNP) complex composition for insertion of a transgene, comprising: (I) a MAD7 nuclease; (II) a guide RNA (gRNA) specific for the MAD7 nuclease, wherein the gRNA comprises a guide sequence capable of hybridizing to a target sequence of the AAVS1, B2M, CIITA, NKG2A, TRAC, CD70, CD38, CD33, or CLYBL loci in a cell (e.g., iPSC), wherein the guide sequence is selected from SEQ ID NOs: 120-130, wherein when the gRNA is complexed with the MAD7 nuclease, the guide sequence directs sequence-specific binding of the MAD7 nuclease to the target sequence, and (III) a transgene vector comprising: (1) left and right polynucleotide sequences that are homologous to the left and right arms of the target sequence of the AAVS1, B2M, CIITA, NKG2A, TRAC, CD70, CD38, CD33, or CLYBL loci, (2) a promoter which is operably linked to (3) a polynucleotide sequence encoding the transgene, and (4) a transcription terminator sequence.

In another aspect, provided herein is a MAD7/gRNA ribonucleoprotein (RNP) complex composition for insertion of a transgene, comprising: (I) a MAD7 nuclease system, wherein the system is encoded by one or more vectors comprising (a) a sequence encoding a guide RNA (gRNA) operably, wherein the sequence is linked to a first regulatory element, wherein the gRNA comprises a guide sequence capable of hybridizing to a target sequence of the AAVS1, B2M, CIITA, NKG2A, TRAC, CD70, CD38, CD33, or CLYBL loci in a cell (e.g., iPSC), wherein the guide sequence is selected from SEQ ID NOs: 120-130, wherein when transcribed, the guide sequence directs sequence-specific binding of the MAD7 complex to the target sequence, and (b) a sequence encoding a MAD7 nuclease, wherein the sequence is operably linked to a second regulatory element, and (II) a transgene vector comprising: (1) left and right polynucleotide sequences that are homologous to the left and right arms of the target sequence of the AAVS1, B2M, CIITA, NKG2A, TRAC, CD70, CD38, CD33, or CLYBL loci, (2) a promoter which is operably linked to (3) a polynucleotide encoding the transgene, and (4) a transcription terminator sequence.

In another aspect, provided herein is a MAD7/gRNA ribonucleoprotein (RNP)-based vector system, comprising: (I) one or more vectors comprising (a) a sequence encoding a guide RNA (gRNA), wherein the sequence is operably linked to a first regulatory element, wherein the gRNA comprises a guide sequence capable of hybridizing to a target sequence of the AAVS1, B2M, CIITA, NKG2A, TRAC, CD70, CD38, CD33, or CLYBL loci in a cell (e.g., iPSC), wherein the guide sequence is selected from SEQ ID NOs: 120-130, wherein when transcribed, the guide sequence directs sequence-specific binding of the MAD7 complex to the target sequence; (b) a sequence encoding a MAD7 nuclease, wherein the sequence is operably linked to a second regulatory element; and (II) a transgene vector comprising: (1) left and right polynucleotide sequences that are homologous to the left and right arms of the target sequence of the AAVS1, B2M, CIITA, NKG2A, TRAC, CD70, CD38, CD33, or CLYBL loci, (2) a promoter which is operably linked to (3) a polynucleotide encoding a transgene, and (4) a transcription terminator sequence.

In various embodiments, the first and/or second regulatory element is a promoter. In some embodiments, the first and second regulatory element are the same. In some embodiments, the first and second regulatory element are different.

In some embodiments of the composition or the vector system described herein, the transgene comprises a sequence encoding a chimeric antigen receptor (CAR), optionally wherein the CAR is specific for a tumor antigen associated with glioblastoma, ovarian cancer, cervical cancer, head and neck cancer, liver cancer, prostate cancer, pancreatic cancer, renal cell carcinoma, bladder cancer, or a hematologic malignancy.

In some embodiments, the guide sequence is specific for the AAVS1 locus. In some embodiments, the gRNA guide sequence specific for the AAVS1 locus comprises SEQ ID NO: 120.

In some embodiments of the composition or the vector system described herein, the transgene comprises a sequence encoding a chimeric antigen receptor (CAR), optionally wherein the CAR is specific for a tumor antigen associated with glioblastoma, ovarian cancer, cervical cancer, head and neck cancer, liver cancer, prostate cancer, pancreatic cancer, renal cell carcinoma, bladder cancer, or a hematologic malignancy and the guide sequence is specific for the AAVS1 locus. In some embodiments, the gRNA guide sequence specific for the AAVS1 locus comprises SEQ ID NO: 120.

In some embodiments of the composition or the vector system described herein, the transgene comprises a sequence encoding an artificial cell death polypeptide.

In some embodiments, the guide sequence is specific for the B2M or CIITA locus. In some embodiments, the gRNA guide sequence is specific for the B2M locus and comprises SEQ ID NO: 121. In some embodiments, the gRNA guide sequence is specific for the CIITA locus and comprises SEQ ID NO: 122 or 126.

In some embodiments of the composition or the vector system described herein, the transgene comprises a sequence encoding an artificial cell death polypeptide and the guide sequence is specific for the B2M or CIITA locus. In some embodiments, the gRNA guide sequence is specific for the B2M locus and comprises SEQ ID NO: 121. In some embodiments, the gRNA guide sequence is specific for the CIITA locus and comprises SEQ ID NO: 122 or 126.

In some embodiments of the composition or the vector system described herein, the transgene comprises a sequence encoding an exogenous cytokine.

In some embodiments, the guide sequence is specific for the B2M or CIITA locus. In some embodiments, the gRNA guide sequence is specific for the B2M locus and comprises SEQ ID NO: 121.

In some embodiments of the composition or the vector system described herein, the transgene comprises a sequence encoding an exogenous cytokine and the guide sequence is specific for the B2M or CIITA locus. In some embodiments, the gRNA guide sequence is specific for the B2M locus and comprises SEQ ID NO: 121

In some embodiments of the composition or the vector system described herein, the gRNA guide sequence is specific for the CIITA locus. In one embodiment, the gRNA guide sequence comprises SEQ ID NO: 122 or 126.

In some embodiments of the composition or the vector system described herein, the gRNA guide sequence is specific for the NKG2A locus. In one embodiment, the gRNA guide sequence comprises SEQ ID NO: 124.

In some embodiments of the composition or the vector system described herein, the gRNA guide sequence is specific for the TRAC locus. In one embodiment, the gRNA guide sequence comprises SEQ ID NO: 125.

In some embodiments of the composition or the vector system described herein, the gRNA guide sequence is specific for the CLYBL locus. In one embodiment, the gRNA guide sequence comprises SEQ ID NO: 123.

In some embodiments of the composition or the vector system described herein, the gRNA guide sequence is specific for the CD70 locus. In one embodiment, the gRNA guide sequence comprises SEQ ID NO: 127.

In some embodiments of the composition or the vector system described herein, the gRNA guide sequence is specific for the CD38 locus. In one embodiment, the gRNA guide sequence comprises SEQ ID NO: 128.

In some embodiments of the composition or the vector system described herein, the gRNA guide sequence is specific for the CD33 locus. In one embodiment, the gRNA guide sequence comprises SEQ ID NO: 129 or 130.

In some embodiments of the composition or the vector system described herein, the left and right polynucleotide sequences that are homologous to the left and right arms of the target sequence of the AAVS1 comprise the nucleotide sequence of SEQ ID NOs: 60 and 61, respectively, or a fragment thereof.

In some embodiments of the composition or the vector system described herein, the left and right polynucleotide sequences that are homologous to the left and right arms of the target sequence of the B2M comprise the nucleotide sequence of SEQ ID NOs: 63 and 64, respectively, or a fragment thereof.

In some embodiments of the composition or the vector system described herein, the left and right polynucleotide sequences that are homologous to the left and right arms of the target sequence of the CIITA comprise the nucleotide sequence of (i) SEQ ID NOs: 66 and 67, respectively, or a fragment thereof, or (ii) SEQ ID NOs: 106 and 107, respectively, or a fragment thereof.

In some embodiments of the composition or the vector system described herein, the left and right polynucleotide sequences that are homologous to the left and right arms of the target sequence of the CLYBL comprise the nucleotide sequence of SEQ ID NOs: 69 and 70, respectively, or a fragment thereof.

In some embodiments of the composition or the vector system described herein, the left and right polynucleotide sequences that are homologous to the left and right arms of the target sequence of the CD70 comprise the nucleotide sequence of SEQ ID NOs: 109 and 110, respectively, or a fragment thereof.

In some embodiments of the composition or the vector system described herein, the left and right polynucleotide sequences that are homologous to the left and right arms of the target sequence of the NKG2A comprise the nucleotide sequence of SEQ ID NOs: 72 and 73, respectively, or a fragment thereof.

In some embodiments of the composition or the vector system described herein, the left and right polynucleotide sequences that are homologous to the left and right arms of the target sequence of the TRAC comprise the nucleotide sequence of SEQ ID NOs: 75 and 76, respectively, or a fragment thereof.

In some embodiments of the composition or the vector system described herein, when the RNP complex is introduced into a cell, expression of the endogenous gene comprising the target sequence complementary to the guide sequence of the gRNA molecule is reduced or eliminated in said cell.

In another aspect, provided herein is one or more retroviruses comprising the vector system described herein.

In another aspect, provided herein is an iPSC transformed with a transgene by the MAD7/gRNA ribonucleoprotein (RNP) composition described herein.

In another aspect, provided herein is an iPSC transformed with the vector system described herein or the one or more retroviruses described herein.

In some embodiments of the iPSC described herein, the transgene comprises a sequence encoding a chimeric antigen receptor (CAR). The CAR may be specific for a tumor antigen associated with glioblastoma, ovarian cancer, cervical cancer, head and neck cancer, liver cancer, prostate cancer, pancreatic cancer, renal cell carcinoma, bladder cancer, or hematologic malignancy. In some embodiments, the tumor antigen associated with glioblastoma is selected from HER2, EGFRvIII, EGFR, CD133, PDGFRA, FGFR1, FGFR3, MET, CD70, ROBO1 and IL13Rα2, the tumor antigen associated with ovarian cancer is selected from FOLR1, FSHR, MUC16, MUC1, Mesothelin, CA125, EpCAM, EGFR, PDGFRα, Nectin-4, and B7H4, the tumor antigen associated with cervical cancer or head and neck cancer is selected from GD2, MUC1, Mesothelin, HER2, and EGFR, the tumor antigen associated with liver cancer is selected from Claudin 18.2, GPC-3, EpCAM, cMET, and AFP, the tumor antigen associated with hematological malignancies is selected from CD19, CD22, CD79, BCMA, GPRC5D, SLAM F7, CD33, CLL1, CD123, and CD70, and the tumor antigen associated with bladder cancer is selected from Nectin-4 and SLITRK6.

In some embodiments of the iPSC described herein, the CAR may be specific for a tumor antigen that is selected from alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker and an oncogene product.

In one embodiment of the iPSCs described herein, the tumor antigen is CD19.

In another aspect, provided herein is an engineered immune-effector cell, or a population thereof, derived from an iPSC described herein. In some embodiments, the immune effector cell is a T cell or NK cell. In some embodiments, the T cell is a CD4+ T cell, a CD8+ T cell, or a combination thereof.

In another aspect, provided herein is a pharmaceutical composition comprising the immuno-effector cell derived from an iPSC described herein.

In another aspect, provided herein is a method for preventing or treating a cancer, the method comprising administering, to an individual in need thereof, a pharmaceutically effective amount of the immune-effector cell or the population described herein, or the pharmaceutical composition described herein. In some embodiments, the cancer is selected from the group consisting of lung cancer, pancreatic cancer, liver cancer, melanoma, bone cancer, breast cancer, colon cancer, leukemia, uterine cancer, ovarian cancer, lymphoma, and brain cancer.

In another aspect, provided herein is a gRNA comprising a guide sequence selected from the group consisting of SEQ ID NOs: 120-130. In some embodiments, the gRNA comprises a guide sequence of SEQ ID NOs: 123, 124, or 125. In one embodiment, the gRNA comprises a guide sequence of SEQ ID NO: 123. In one embodiment, the gRNA comprises a guide sequence of SEQ ID NO: 124. In one embodiment, the gRNA comprises a guide sequence of SEQ ID NO: 125.

### BRIEF DESCRIPTION OF THE DRAWINGS

FIG. 1 depicts an AAVS1 targeting vector map.

FIG. 2 depicts a B2M targeting vector map.

FIG. 3 depicts a CIITA targeting vector map.

FIG. 4 depicts a CLYBL targeting vector map.

FIG. 5 depicts a NKG2A targeting vector map.

FIG. 6 depicts a TRAC targeting vector map.

FIGS. 7A-7C depict flow cytometry analysis of cells engineered with a CAR transgene inserted at the AAVS1 site. FIG. 7A depicts flow cytometry analysis of bulk population of cells post-engineering. FIG. 7B depicts flow cytometry analysis of cells post-sorting for CAR positive cells. FIG. 7C depicts flow cytometry analysis of CAR positive single cell clones.

FIGS. 8A-8C depict flow cytometry analysis of cells engineered with an HLA-E transgene inserted at the B2M site. FIG. 8A depicts flow cytometry analysis of bulk population of cells post-engineering. FIG. 8B depicts flow cytometry analysis of cells post-sorting for HLA-E positive, B2M negative cells. FIG. 8C depicts flow cytometry analysis of HLA-E positive, B2M negative single cell clones.

FIGS. 9A-9C depict flow cytometry analysis of cells engineered with an EGFR transgene inserted at the CIITA site. FIG. 9A depicts flow cytometry analysis of bulk population of cells post-engineering. FIG. 9B depicts flow cytometry analysis of cells post-sorting for EGFR cells. FIG. 9C depicts flow cytometry analysis of EGFR positive single cell clones.

FIGS. 10A-10B depict flow cytometry analysis of cells engineered with a PSMA transgene inserted at the CLYBL site. FIG. 10A depicts flow cytometry analysis of bulk population of cells post-engineering. FIG. 10B depicts flow cytometry analysis of cells post-sorting for PSMA positive cells.

FIGS. 11A-11B depict flow cytometry analysis of cells engineered with an IL15-IL15RA transgene inserted at the NKG2A site. FIG. 11A depicts flow cytometry analysis of bulk population of cells post-engineering. FIG. 11B depicts flow cytometry analysis of cells post-sorting for IL15-IL15RA positive cells.

FIG. 12 depicts an CIITA targeting vector map.

FIG. 13 depicts an CD70 targeting vector map.

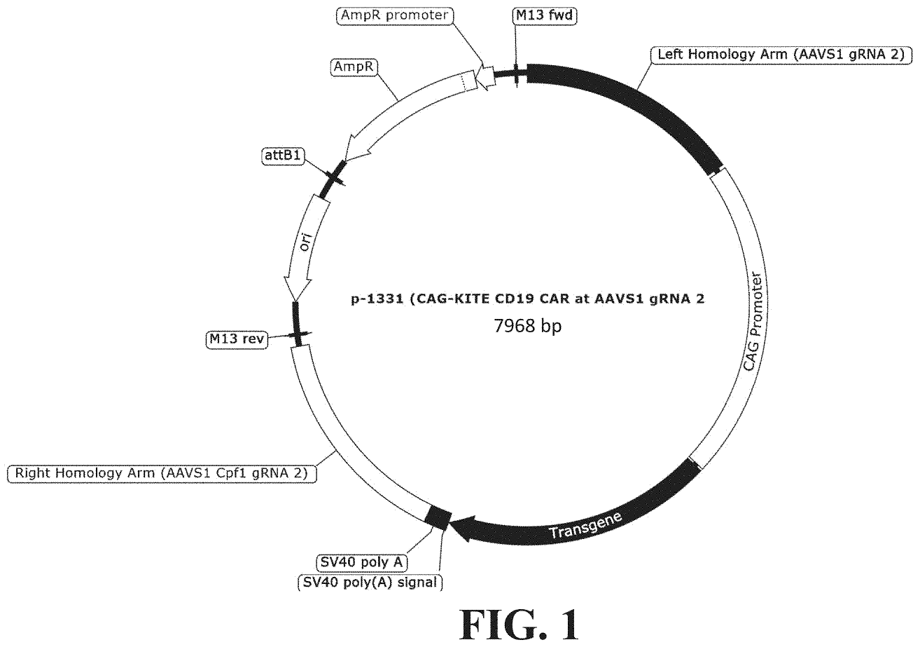

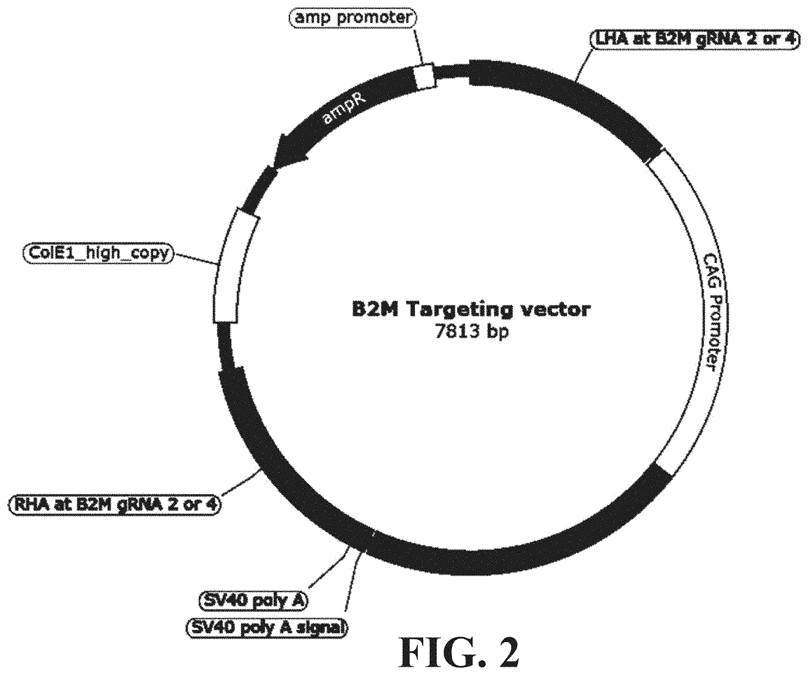

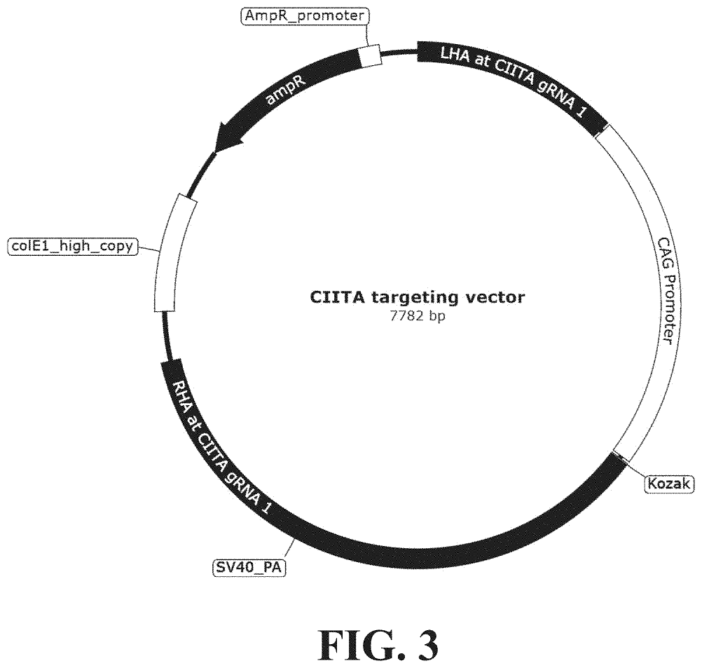

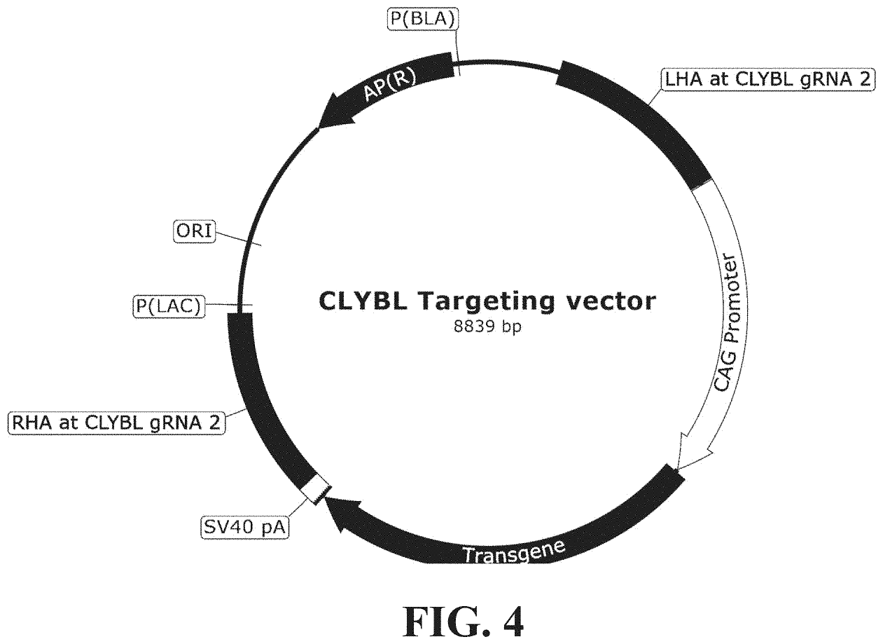

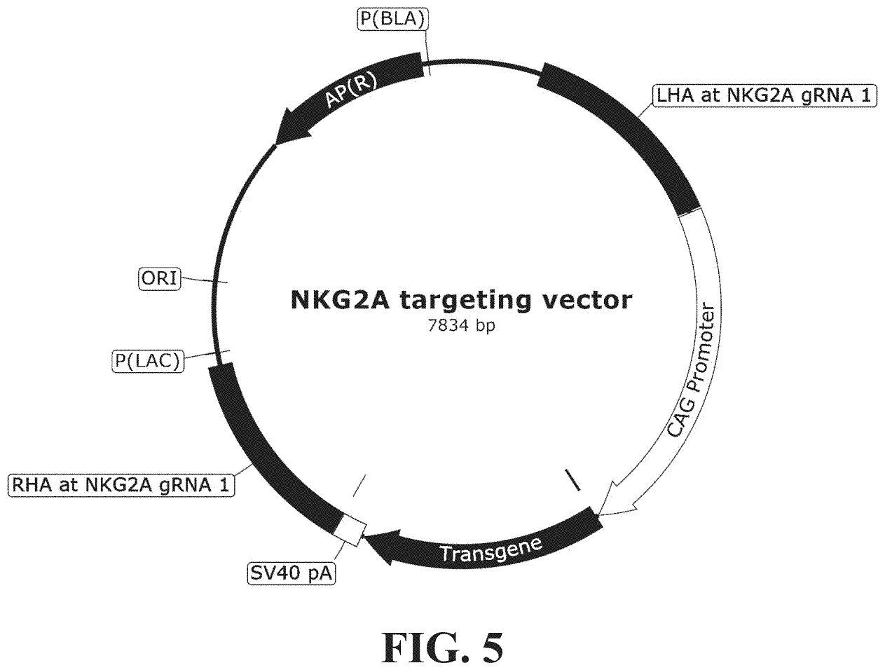

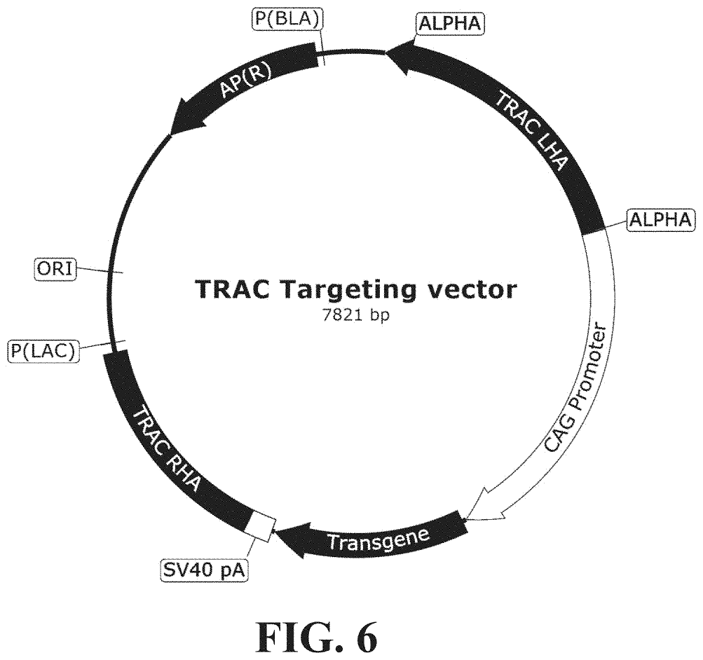

### DETAILED DESCRIPTION

The present application provides, among other things, compositions and methods for use in genome engineering of cells, such as iPSCs. Specifically, the methods and compositions described relate to introducing nucleic acids encoding transgenes into iPSCs such as pluripotent hematopoietic stem cells and/or progenitor cells (HSC/PC) and preparing immune-effector cells such as T cells, NK cells, macrophages and dendritic cells derived from iPSCs. Specifically, disclosed are DNA sequences encoding gene transfer vectors for the genomic engineering of human cell lines and the methods used. The gene transfer vectors are designed for inserting transgenes into the AAVS1, B2M, CIITA, NKG2A, TRAC, CD70, CD38, CD33, and/or CLYBL loci of human cells (e.g., iPSC) and include promoter sequences, terminator sequences and homology arms specific for the loci in question. The gene transfer vectors can be used with a CRISPR nuclease-based system, such as the MAD7 nuclease-based system. Also included are novel guide sequences for use with CRISPR nuclease-based systems for insertion of the transgenes, particularly with the MAD7 nuclease-based system. In some embodiments, MAD7 nuclease-based system includes a non-naturally occurring or engineered MAD7 nuclease.

### I. Definitions

Various publications, articles and patents are cited or described in the background and throughout the specification; each of these references is herein incorporated by reference in its entirety for all intended purposes. Discussion of documents, acts, materials, devices, articles or the like which has been included in the present specification is for the purpose of providing context for embodiments of the present disclosure. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic (e.g., concentration or concentration range) of a component of the invention, indicate that the described dimension/characteristic is not a strict boundary or parameter and does not exclude minor variations therefrom that are functionally the same or similar, as would be understood by one having ordinary skill in the art. Unless otherwise stated, any numerical values, such as a concentration or a concentration range described herein, are to be understood as being modified in all instances by the term “about.” At a minimum, such references that include a numerical parameter would include variations that, using mathematical and industrial principles accepted in the art (e.g., rounding, measurement or other systematic errors, manufacturing tolerances, etc.), would not vary the least significant digit. In some embodiments, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

The terms “identical” or percent “identity,” in the context of two or more nucleic acids (e.g., guide RNA sequences or homology arm sequences) or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc. (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as “oligonucleotides”.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” or “insertion” it is meant that one or more sequences or nucleotides of an exogenous construct is stably inserted into the cellular genome, i.e., covalently linked to the nucleic acid sequence within the cell's chromosomal or mitochondrial DNA. By “targeted integration” it is meant that the nucleotide(s) of a construct is inserted into the cell's chromosomal or mitochondrial DNA at a pre-selected site or “integration site”. The term “integration” or “insertion” as used herein further refers to a process involving insertion of one or more sequences or nucleotides of the exogenous construct, with or without deletion of an endogenous sequence or one or more nucleotides at the integration site. In the case, where there is a deletion at the insertion site, “integration” can further comprise replacement of the endogenous sequence or one or more nucleotides that are deleted with the one or more inserted sequences or nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term “endogenous” when used in reference to expression of an encoding nucleic acid refers to expression of an encoding nucleic acid natively contained within the cell and not exogenously introduced.

As used herein, a “transgene”, “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably linked” refers to the operational linkage of nucleic acid sequences or amino acid sequences so that they are placed in functional relationships with each other. For example, a promoter is operably linked with a coding sequence or functional RNA when it is capable of affecting the expression of that coding sequence or functional RNA (i.e., the coding sequence or functional RNA is under the transcriptional control of the promoter). Coding sequences can be operably linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptides (e.g., CAR) can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### II. Induced Pluripotent Stem Cells (IPSCs) and Immune Effector Cells

IPSCs have unlimited self-renewing capacity. Use of iPSCs enables cellular engineering to produce a controlled cell bank of modified cells that can be expanded and differentiated into desired immune effector cells, supplying large amounts of homogeneous allogeneic therapeutic products.

Provided herein are genetically engineered iPSCs and derivative cells thereof. The selected genomic modifications provided herein enhance the therapeutic properties of the derivative cells. The derivative cells are functionally improved and suitable for allogenic off-the-shelf cell therapies following a combination of selective modalities being introduced to the cells at the level of iPSC through genomic engineering. This approach can help to reduce the side effects mediated by cytokine release syndrome CRS/graft-versus-host disease (GVHD) and prevent long-term autoimmunity while providing excellent efficacy.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells).

As used herein, the term “immune cell” or “immune-effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the term “engineered immune cell” or “engineered immune-effector cell” refers to an immune cell that has been genetically modified by the addition of exogenous genetic material in the form of DNA or RNA to the total genetic material of the cell.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells; γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 or CD16 and the absence of the T cell receptor (CD3). The NK cell can also refer to a genetically engineered NK cell, such as a NK cell modified to express a chimeric antigen receptor (CAR). The NK cell can also be differentiated from a stem cell or progenitor cell.

The induced pluripotent stem cell (iPSC) parental cell lines may be generated from peripheral blood mononuclear cells (PBMCs) or T-cells using any known method for introducing re-programming factors into non-pluripotent cells such as the episomal plasmid-based process as previously described in U.S. Pat. Nos. 8,546,140; 9,644,184; 9,328,332; and 8,765,470, the complete disclosures of which are incorporated herein by reference in their entirety for all intended purposes. The reprogramming factors may be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSCs are clonal iPSCs or are obtained from a pool of iPSCs and the genome edits are introduced by making one or more targeted integration and/or in/del at one or more selected sites. In another embodiment, the iPSCs are obtained from human T cells having antigen specificity and a reconstituted TCR gene (hereinafter, also refer to as “T-iPS” cells) as described in U.S. Pat. Nos. 9,206,394, and 10,787,642 hereby incorporated by reference into the present application in their entirety for all intended purposes.

Derivative Immune Effector Cells

In another aspect, this disclosure relates to a cell derived from differentiation of an iPSC, a derivative immune effector cell. As described above, the genomic edits introduced into the iPSC are retained in the derivative immune effector cell. In certain embodiments of the derivative cell obtained from iPSC differentiation, the derivative cell is a hematopoietic cell, including, but not limited to, HSCs (hematopoietic stem and progenitor cells), hematopoietic multipotent progenitor cells, T cell progenitors, NK cell progenitors, T cells, NKT cells, NK cells, and B cells. In certain embodiments, the derivative cell is an immune effector cell, such as a NK cell or a T cell.

In certain embodiments, the application provides a natural killer (NK) cell or a T cell derived from an iPSC with one or more transgene inserts prepared in accordance with this disclosure.

Also provided is a method of manufacturing the derivative cell. The method comprises differentiating the iPSC under conditions for cell differentiation to thereby obtain the derivative cell.

An iPSC of the application can be differentiated by any method known in the art. Exemplary methods are described in U.S. Pat. Nos. 8,846,395, 8,945,922, 8,318,491, and Int. Pat. Publ. Nos. WO2010/099539, WO2012/109208, WO2017/070333, WO2017/179720, WO2016/010148, WO2018/048828 and WO2019/157597, each of which are herein incorporated by reference in its entirety for all intended purposes.

### III. Targeted Genome Editing at Selected Locus in iPSCs

According to embodiments of the application, one or more of the exogenous polynucleotides are inserted at one or more loci on one or more chromosomes of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein are the terms “targeted integration” and “targeted insertion”, referring to a process involving insertion of one or more exogenous sequences at pre-selected sites in the genome, with or without deletion of an endogenous sequence at the insertion site.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration”.

Targeted nucleases include naturally occurring and recombinant nucleases such as CRISPR related nucleases from families including Cas, Cpf, Cse, Csy, Csn, Csd, Cst, Csh, Csa, Csm, and Cmr; restriction endonucleases; meganucleases; homing endonucleases, and the like. As an example, CRISPR/Cpf1 comprises two major components: (1) a Cpf1 endonuclease and (2) a guide nucleic acid, which can be DNA or RNA. When co-expressed, the two components form a ribonucleoprotein (RNP) complex that is recruited to a target DNA sequence comprising PAM and a seeding region near PAM. The guide nucleic acid can be used to guide Cpf1 to target selected sequences. These two components can then be delivered to mammalian cells via transfection or transduction.

One type of alternative CRISPR nuclease family, Cpf1 (also known as Cas12a), has been used for genome editing since the first report in 2015 (Zetsche et al Cell, 163(3), 759-771). Cpf1 nucleases exhibit different characteristics to Cas9 nucleases, such as a staggered DSB, a T-rich PAM and the native use of only 1 guide RNA molecule to form a complex with Cpf1 and target the DNA. These characteristics enable Cpf1 nucleases to be used in target organisms or regions within an organism's genome where a lower GC content makes the use of Cas9 less feasible.

Recently, an alternative CRISPR nuclease referred to as MAD7 has been disclosed in U.S. Pat. Nos. 9,982,279 and 10,337,028, the contents of which are hereby incorporated in their entirety for all intended purposes. The company Inscripta has made this nuclease free for all commercial or academic research. As such, its use for commercial genome editing is of great interest. Inscripta reports that MAD7 was developed from Eubacterium rectale and has proven its functionality in E. coli, S. cerevisiae and in the human HEK293T cell line. MAD7 has only 31% identity with Acidaminococcus sp. BV3L6 Cpf1 (AsCpf1), to which it also shares a T-rich PAM site (5′-YTTN-3′), and a protospacer (the region of the gRNA which associates the nuclease to the DNA target) length of 21 nucleotides. Certain embodiments of the present disclosure are particularly suitable for use with the endonuclease MAD7. This nuclease only requires a crRNA for gene editing and allows for specific targeting of AT rich regions of the genome. MAD7 cleaves DNA with a staggered cut as compared to S. pyogenes which has blunt cutting.

Exemplary MAD7 sequences and scaffold sequences for guide nucleic acid are provided in Table 1. In general, a “scaffold sequence” includes any sequence that has sufficient sequence to promote formation of a targetable ribonucleoprotein complex. The targetable ribonucleoprotein complex can comprise a nucleic acid-guided nuclease (e.g., MAD7) and a guide nucleic acid comprising a scaffold sequence and a guide sequence. Sufficient sequence within the scaffold sequence to promote formation of a targetable ribonucleoprotein complex may include a degree of complementarity along the length of two sequence regions within the scaffold sequence, such as one or two sequence regions involved in forming a secondary structure (e.g., a pseudoknot region). The one or two sequence regions may be comprised or encoded on the same polynucleotide. Alternatively, the one or two sequence regions may be comprised or encoded on separate polynucleotides. In some embodiments, a scaffold sequence can comprise the sequence of any one of SEQ ID NO: 117-119. In some embodiments, the scaffold sequence comprises the sequence of SEQ ID NO: 117. In some embodiments, the scaffold sequence comprises the sequence of SEQ ID NO: 118. In some embodiments, the scaffold sequence comprises the sequence of SEQ ID NO: 119.

| | | | SEQ ID |
|---|---|---|---|
| | Sequence | | NO |
| WT MAD7 | ATGAATAATG GAACAAATAA CTTTCAGAAT TTTATCGGAA TTTCTTCTTT GCAGAAGACT | 60 | 114 |
| nucleic acid | CTTAGGAATG CTCTCATTCC AACCGAAACA ACACAGCAAT TTATTGTTAA AAACGGAATA | 120 | |
| sequence | ATTAAAGAAG ATGAGCTAAG AGGAGAAAAT CGTCAGATAC TTAAAGATAT CATGGATGAT | 180 | |
| | TATTACAGAG GTTTCATTTC AGAAACTTTA TCGTCAATTG ATGATATTGA CTGGACTTCT | 240 | |
| | TTATTTGAGA AAATGGAAAT TCAGTTAAAA AATGGAGATA ACAAAGACAC TCTTATAAAA | 300 | |
| | GAACAGACTG AATACCGTAA GGCAATTCAT AAAAAATTTG CAAATGATGA TAGATTTAAA | 360 | |
| | AATATGTTCA GTGCAAAATT AATCTCAGAT ATTCTTCCTG AATTTGTCAT TCATAACAAT | 420 | |
| | AATTATTCTG CATCAGAAAA GGAAGAAAAA ACACAGGTAA TTAAATTATT TTCCAGATTT | 480 | |
| | GCAACGTCAT TCAAGGACTA TTTTAAAAAC AGGGCTAATT GTTTTTCGGC TGATGATATA | 540 | |
| | TCTTCATCTT CTTGTCATAG AATAGTTAAT GATAATGCAG AGATATTTTT TAGTAATGCA | 600 | |
| | TTGGTGTATA GGAGAATTGT AAAAAGTCTT TCAAATGATG ATATAAATAA AATATCCGGA | 660 | |
| | GATATGAAGG ATTCATTAAA GGAAATGTCT CTGGAAGAAA TTTATTCTTA TGAAAAATAT | 720 | |
| | GGGGAATTTA TTACACAGGA AGGTATATCT TTTTATAATG ATATATGTGG TAAAGTAAAT | 780 | |
| | TCATTTATGA ATTTATATTG CCAGAAAAAT AAAGAAAACA AAAATCTCTA TAAGCTGCAA | 840 | |
| | AAGCTTCATA AACAGATACT GTGCATAGCA GATACTTCTT ATGAGGTGCC GTATAAATTT | 900 | |
| | GAATCAGATG AAGAGGTTTA TCAATCAGTG AATGGATTTT TGGACAATAT TAGTTCGAAA | 960 | |
| | CATATCGTTG AAAGATTGCG TAAGATTGGA GACAACTATA ACGGCTACAA TCTTGATAAG | 1020 | |
| | ATTTATATTG TTAGTAAATT CTATGAATCA GTTTCACAAA AGACATATAG AGATTGGGAA | 1080 | |
| | ACAATAAATA CTGCATTAGA AATTCATTAC AACAATATAT TACCCGGAAA TGGTAAATCT | 1140 | |
| | AAAGCTGACA AGGTAAAAAA AGCGGTAAAG AATGATCTGC AAAAAAGCAT TACTGAAATC | 1200 | |
| | AATGAGCTTG TTAGCAATTA TAAATTATGT TCGGATGATA ATATTAAAGC TGAGACATAT | 1260 | |
| | ATACATGAAA TATCACATAT TTTGAATAAT TTTGAAGCAC AGGAGCTTAA GTATAATCCT | 1320 | |
| | GAAATTCATC TGGTGGAAAG TGAATTGAAA GCATCTGAAT TAAAAAATGT TCTCGATGTA | 1380 | |
| | ATAATGAATG CTTTTCATTG GTGTTCGGTT TTCATGACAG AGGAGCTGGT AGATAAAGAT | 1440 | |
| | AATAATTTTT ATGCCGAGTT AGAAGAGATA TATGACGAAA TATATCCGGT AATTTCATTG | 1500 | |
| | TATAATCTTG TGCGTAATTA TGTAACGCAG AAGCCATATA GTACAAAAAA AATTAAATTG | 1560 | |
| | AATTTTGGTA TTCCTACACT AGCGGATGGA TGGAGTAAAA GTAAAGAATA TAGTAATAAT | 1620 | |
| | GCAATTATTC TCATGCGTGA TAATTTGTAC TATTTAGGAA TATTTAATGC AAAAAATAAG | 1680 | |
| | CCTGACAAAA AGATAATTGA AGGTAATACA TCAGAAAATA AAGGGGATTA TAAGAAGATG | 1740 | |
| | ATTTATAATC TTCTGCCAGG ACCAAATAAA ATGATCCCCA AGGTATTCCT CTCTTCAAAA | 1800 | |
| | ACCGGAGTGG AAACATATAA GCCGTCTGCC TATATATTGG AGGGCTATAA ACAAAACAAG | 1860 | |
| | CATATTAAAT CCTCTAAGGA TTTTGATATA ACATTTTGTC ACGATTTGAT TGATTATTTT | 1920 | |
| | AAGAACTGTA TAGCAATACA TCCTGAATGG AAGAATTTTG GCTTTGATTT TTCTGACACC | 1980 | |
| | TCCACATATG AAGATATCAG CGGATTTTAC AGAGAAGTCG AATTACAAGG TTATAAAATC | 2040 | |
| | GACTGGACAT ATATCAGCGA AAAGGATATT GATTTGTTGC AGGAAAAAGG ACAGTTATAT | 2100 | |
| | TTATTCCAAA TATATAACAA AGATTTTTCC AAGAAAAGTA CCGGAAATGA TAATCTTCAT | 2160 | |
| | ACTATGTATT TGAAGAATTT GTTTAGTGAA GAGAATTTAA AGGATATTGT ACTGAAATTA | 2220 | |
| | AACGGTGAGG CGGAAATCTT CTTTAGAAAA TCAAGCATAA AGAATCCAAT AATTCATAAA | 2280 | |
| | AAAGGCTCTA TTCTTGTTAA TAGAACATAT GAAGCAGAGG AAAAAGATCA ATTTGGAAAT | 2340 | |
| | ATCCAGATAG TCAGAAAAAA CATACCGGAA AATATATATC AGGAGCTTTA TAAATATTTC | 2400 | |
| | AATGATAAAA GTGATAAAGA ACTTTCGGAT GAAGCAGCTA AGCTTAAGAA TGTAGTAGGT | 2460 | |
| | CATCATGAGG CTGCTACAAA CATAGTAAAA GATTATAGAT ATACATATGA TAAATATTTT | 2520 | |
| | cttcatatgc CTATTACAAT CAATTTTAAA GCCAATAAGA CAGGCTTTAT TAATGACAGA | 2580 | |
| | ATATTACAAT ATATTGCTAA AGAAAAGGAT TTGCATGTAA TAGGCATTGA TCGTGGTGAA | 2640 | |
| | AGAAACCTGA TATATGTTTC AGTAATTGAT ACTTGTGGAA ATATTGTTGA ACAAAAATCG | 2700 | |
| | TTTAACATTG TTAATGGATA TGATTATCAG ATTAAGCTCA AGCAGCAGGA GGGGGCGCGA | 2760 | |
| | CAAATCGCAC GAAAAGAATG GAAAGAAATC GGCAAAATAA AAGAAATTAA AGAAGGCTAT | 2820 | |
| | TTATCTCTTG TAATTCATGA AATTTCAAAG ATGGTTATTA AATATAATGC CATAATTGCA | 2880 | |
| | ATGGAGGATT TAAGCTACGG ATTTAAAAAA GGTCGTTTCA AGGTTGAGCG ACAGGTTTAC | 2940 | |
| | CAGAAGTTTG AGACAATGCT TATCAACAAA CTCAACTATC TGGTATTTAA AGATATATCC | 3000 | |
| | ATAACGGAAA ACGGTGGTCT TCTAAAGGGA TACCAGCTTA CATATATTCC AGATAAACTG | 3060 | |
| | AAAAATGTGG GTCATCAATG TGGCTGTATA TTTTATGTAC CTGCTGCCTA TACATCAAAA | 3120 | |
| | ATAGATCCTA CAACCGGATT TGTAAATATA TTCAAATTTA AAGATTTAAC AGTTGATGCG | 3180 | |
| | AAGAGAGAAT TTATAAAAAA ATTTGACAGT ATCAGATATG ATTCAGAAAA AAATCTGTTT | 3240 | |
| | TGTTTTACAT TCGATTATAA TAACTTTATT ACGCAAAATA CTGTTATGTC AAAGTCAAGC | 3300 | |
| | TGGAGTGTAT ATACGTACGG AGTTAGGATA AAAAGAAGAT TTGTCAATGG CAGGTTCTCA | 3360 | |
| | AATGAATCGG ATACAATTGA TATAACAAAA GATATGGAAA AAACACTCGA AATGACAGAT | 3420 | |
| | ATAAATTGGA GAGATGGTCA TGATCTGAGG CAGGATATTA TTGATTATGA AATCGTACAA | 3480 | |
| | CACATATTTG AGATTTTTAG ATTGACTGTA CAAATGAGAA ACAGTTTAAG TGAATTAGAA | 3540 | |
| | GACAGGGATT ATGACCGTTT GATTTCTCCG GTGCTCAATG AAAATAATAT ATTTTATGAT | 3600 | |
| | TCAGCTAAAG CAGGAGATGC GTTACCTAAA GACGCAGATG CTAATGGTGC ATATTGTATA | 3660 | |
| | GCTCTAAAAG GCTTGTATGA AATCAAACAA ATTACAGAGA ATTGGAAAGA AGACGGTAAG | 3720 | |
| | TTTTCAAGAG ATAAACTTAA AATTTCCAAT AAGGACTGGT TTGACTTTAT TCAAAATAAA | 3780 | |
| | AGGTATTTAT AA | 3792 | |
| Codon | ATGAACAACG GCACAAATAA TTTTCAGAAC TTCATCGGGA TCTCAAGTTT GCAGAAAACG | 60 | 115 |
| optimized | CTGCGCAATG CTCTGATCCC CACGGAAACC ACGCAACAGT TCATCGTCAA GAACGGAATA | 120 | |
| nucleic acid | ATTAAAGAAG ATGAGTTACG TGGCGAGAAC CGCCAGATTC TGAAAGATAT CATGGATGAC | 180 | |
| sequence | TACTACCGCG GATTCATCTC TGAGACTCTG AGTTCTATTG ATGACATAGA TTGGACTAGC | 240 | |
| | CTGTTCGAAA AAATGGAAAT TCAGCTGAAA AATGGTGATA ATAAAGATAC CTTAATTAAG | 300 | |
| | GAACAGACAG AGTATCGGAA AGCAATCCAT AAAAAATTTG CGAACGACGA TCGGTTTAAG | 360 | |
| | AACATGTTTA GCGCCAAACT GATTAGTGAC ATATTACCTG AATTTGTCAT CCACAACAAT | 420 | |
| | AATTATTCGG CATCAGAGAA AGAGGAAAAA ACCCAGGTGA TAAAATTGTT TTCGCGCTTT | 480 | |
| | GCGACTAGCT TTAAAGATTA CTTCAAGAAC CGTGCAAATT GCTTTTCAGC GGACGATATT | 540 | |
| | TCATCAAGCA GCTGCCATCG CATCGTCAAC GACAATGCAG AGATATTCTT TTCAAATGCG | 600 | |
| | CTGGTCTACC GCCGGATCGT AAAATCGCTG AGCAATGACG ATATCAACAA AATTTCGGGC | 660 | |
| | GATATGAAAG ATTCATTAAA AGAAATGAGT CTGGAAGAAA TATATTCTTA CGAGAAGTAT | 720 | |
| | GGGGAATTTA TTACCCAGGA AGGCATTAGC TTCTATAATG ATATCTGTGG GAAAGTGAAT | 780 | |
| | TCTTTTATGA ACCTGTATTG TCAGAAAAAT AAAGAAAACA AAAATTTATA CAAACTTCAG | 840 | |
| | AAACTTCACA AACAGATTCT ATGCATTGCG GACACTAGCT ATGAGGTCCC GTATAAATTT | 900 | |
| | GAAAGTGACG AGGAAGTGTA CCAATCAGTT AACGGCTTCC TTGATAACAT TAGCAGCAAA | 960 | |
| | CATATAGTCG AAAGATTACG CAAAATCGGC GATAACTATA ACGGCTACAA CCTGGATAAA | 1020 | |
| | ATTTATATCG TGTCCAAATT TTACGAGAGC GTTAGCCAAA AAACCTACCG CGACTGGGAA | 1080 | |
| | ACAATTAATA CCGCCCTCGA AATTCATTAC AATAATATCT TGCCGGGTAA CGGTAAAAGT | 1140 | |
| | AAAGCCGACA AAGTAAAAAA AGCGGTTAAG AATGATTTAC AGAAATCCAT CACCGAAATA | 1200 | |
| | AATGAACTAG TGTCAAACTA TAAGCTGTGC AGTGACGACA ACATCAAAGC GGAGACTTAT | 1260 | |
| | ATACATGAGA TTAGCCATAT CTTGAATAAC TTTGAAGCAC AGGAATTGAA ATACAATCCG | 1320 | |
| | GAAATTCACC TAGTTGAATC CGAGCTCAAA GCGAGTGAGC TTAAAAACGT GCTGGACGTG | 1380 | |
| | ATCATGAATG CGTTTCATTG GTGTTCGGTT TTTATGACTG AGGAACTTGT TGATAAAGAC | 1440 | |
| | AACAATTTTT ATGCGGAACT GGAGGAGATT TACGATGAAA TTTATCCAGT AATTAGTCTG | 1500 | |
| | TACAACCTGG TTCGTAACTA CGTTACCCAG AAACCGTACA GCACGAAAAA GATTAAATTG | 1560 | |
| | AACTTTGGAA TACCGACGTT AGCAGACGGT TGGTCAAAGT CCAAAGAGTA TTCTAATAAC | 1620 | |
| | GCTATCATAC TGATGCGCGA CAATCTGTAT TATCTGGGCA TCTTTAATGC GAAGAATAAA | 1680 | |
| | CCGGACAAGA AGATTATCGA GGGTAATACG TCAGAAAATA AGGGTGACTA CAAAAAGATG | 1740 | |
| | ATTTATAATT TGCTCCCGGG TCCCAACAAA ATGATCCCGA AAGTTTTCTT GAGCAGCAAG | 1800 | |
| | ACGGGGGTGG AAACGTATAA ACCGAGCGCC TATATCCTAG AGGGGTATAA ACAGAATAAA | 1860 | |
| | CATATCAAGT CTTCAAAAGA CTTTGATATC ACTTTCTGTC ATGATCTGAT CGACTACTTC | 1920 | |
| | AAAAACTGTA TTGCAATTCA TCCCGAGTGG AAAAACTTCG GTTTTGATTT TAGCGACACC | 1980 | |
| | AGTACTTATG AAGACATTTC CGGGTTTTAT CGTGAGGTAG AGTTACAAGG TTACAAGATT | 2040 | |
| | GATTGGACAT ACATTAGCGA AAAAGACATT GATCTGCTGC AGGAAAAAGG TCAACTGTAT | 2100 | |
| | CTGTTCCAGA TATATAACAA AGATTTTTCG AAAAAATCAA CCGGGAATGA CAACCTTCAC | 2160 | |
| | ACCATGTACC TGAAAAATCT TTTCTCAGAA GAAAATCTTA AGGATATCGT CCTGAAACTT | 2220 | |
| | AACGGCGAAG CGGAAATCTT CTTCAGGAAG AGCAGCATAA AGAACCCAAT CATTCATAAA | 2280 | |
| | AAAGGCTCGA TTTTAGTCAA CCGTACCTAC GAAGCAGAAG AAAAAGACCA GTTTGGCAAC | 2340 | |
| | ATTCAAATTG TGCGTAAAAA TATTCCGGAA AACATTTATC AGGAGCTGTA CAAATACTTC | 2400 | |
| | AACGATAAAA GCGACAAAGA GCTGTCTGAT GAAGCAGCCA AACTGAAGAA TGTAGTGGGA | 2460 | |
| | CACCACGAGG CAGCGACGAA TATAGTCAAG GACTATCGCT ACACGTATGA TAAATACTTC | 2520 | |
| | CTTCATATGC CTATTACGAT CAATTTCAAA GCCAATAAAA CGGGTTTTAT TAATGATAGG | 2580 | |
| | ATCTTACAGT ATATCGCTAA AGAAAAAGAC TTACATGTGA TCGGCATTGA TCGGGGCGAG | 2640 | |
| | CGTAACCTGA TCTACGTGTC CGTGATTGAT ACTTGTGGTA ATATAGTTGA ACAGAAAAGC | 2700 | |
| | TTTAACATTG TAAACGGCTA CGACTATCAG ATAAAACTGA AACAACAGGA GGGCGCTAGA | 2760 | |
| | CAGATTGCGC GGAAAGAATG GAAAGAAATT GGTAAAATTA AAGAGATCAA AGAGGGCTAC | 2820 | |
| | CTGAGCTTAG TAATCCACGA GATCTCTAAA ATGGTAATCA AATACAATGC AATTATAGCG | 2880 | |
| | ATGGAGGATT TGTCTTATGG TTTTAAAAAA GGGCGCTTTA AGGTCGAACG GCAAGTTTAC | 2940 | |
| | CAGAAATTTG AAACCATGCT CATCAATAAA CTCAACTATC TGGTATTTAA AGATATTTCG | 3000 | |
| | ATTACCGAGA ATGGCGGTCT CCTGAAAGGT TATCAGCTGA CATACATTCC TGATAAACTT | 3060 | |
| | AAAAACGTGG GTCATCAGTG CGGCTGCATT TTTTATGTGC CTGCTGCATA CACGAGCAAA | 3120 | |
| | ATTGATCCGA CCACCGGCTT TGTGAATATC TTTAAATTTA AAGACCTGAC AGTGGACGCA | 3180 | |
| | AAACGTGAAT TCATTAAAAA ATTTGACTCA ATTCGTTATG ACAGTGAAAA AAATCTGTTC | 3240 | |
| | TGCTTTACAT TTGACTACAA TAACTTTATT ACGCAAAACA CGGTCATGAG CAAATCATCG | 3300 | |
| | TGGAGTGTGT ATACATACGG CGTGCGCATC AAACGTCGCT TTGTGAACGG CCGCTTCTCA | 3360 | |
| | AACGAAAGTG ATACCATTGA CATAACCAAA GATATGGAGA AAACGTTGGA AATGACGGAC | 3420 | |
| | ATTAACTGGC GCGATGGCCA CGATCTTCGT CAAGACATTA TAGATTATGA AATTGTTCAG | 3480 | |
| | CACATATTCG AAATTTTCCG TTTAACAGTG CAAATGCGTA ACTCCTTGTC TGAACTGGAG | 3540 | |
| | GACCGTGATT ACGATCGTCT CATTTCACCT GTACTGAACG AAAATAACAT TTTTTATGAC | 3600 | |
| | AGCGCGAAAG CGGGGGATGC ACTTCCTAAG GATGCCGATG CAAATGGTGC GTATTGTATT | 3660 | |
| | GCATTAAAAG GGTTATATGA AATTAAACAA ATTACCGAAA ATTGGAAAGA AGATGGTAAA | 3720 | |
| | TTTTCGCGCG ATAAACTCAA AATCAGCAAT AAAGATTGGT TCGACTTTAT CCAGAATAAG | 3780 | |
| | CGCTATCTCT AA | 3792 | |
| Amino acid | MNNGTNNFQN FIGISSLQKT LRNALIPTET TQQFIVKNGI IKEDELRGEN RQILKDIMDD | 60 | 116 |
| sequence | YYRGFISETL SSIDDIDWTS LFEKMEIQLK NGDNKDTLIK EQTEYRKAIH KKFANDDRFK | 120 | |
| | NMFSAKLISD ILPEFVIHNN NYSASEKEEK TQVIKLFSRF ATSFKDYFKN RANCFSADDI | 180 | |
| | SSSSCHRIVN DNAEIFFSNA LVYRRIVKSL SNDDINKISG DMKDSLKEMS LEEIYSYEKY | 240 | |
| | GEFITQEGIS FYNDICGKVN SFMNLYCQKN KENKNLYKLQ KLHKQILCIA DTSYEVPYKF | 300 | |
| | ESDEEVYQSV NGFLDNISSK HIVERLRKIG DNYNGYNLDK IYIVSKFYES VSQKTYRDWE | 360 | |
| | TINTALEIHY NNILPGNGKS KADKVKKAVK NDLQKSITEI NELVSNYKLC SDDNIKAETY | 420 | |
| | IHEISHILNN FEAQELKYNP EIHLVESELK ASELKNVLDV IMNAFHWCSV FMTEELVDKD | 480 | |
| | NNFYAELEEI YDEIYPVISL YNLVRNYVTQ KPYSTKKIKL NFGIPTLADG WSKSKEYSNN | 540 | |
| | AIILMRDNLY YLGIFNAKNK PDKKIIEGNT SENKGDYKKM IYNLLPGPNK MIPKVFLSSK | 600 | |
| | TGVETYKPSA YILEGYKQNK HTKSSKDFDT TFCHDLIDYF KNCTAIHPEW KNFGFDFSDT | 660 | |
| | STYEDISGFY REVELQGYKI DWTYISEKDI DLLQEKGQLY LFQIYNKDFS KKSTGNDNLH | 720 | |
| | TMYLKNLFSE ENLKDIVLKL NGEAEIFFRK SSIKNPIIHK KGSILVNRTY EAEEKDQFGN | 780 | |
| | IQIVRKNIPE NIYQELYKYF NDKSDKELSD EAAKLKNVVG HHEAATNIVK DYRYTYDKYF | 840 | |
| | LHMPITINFK ANKTGFINDR ILQYIAKEKD LHVIGIDRGE RNLIYVSVID TCGNIVEQKS | 900 | |
| | FNIVNGYDYQ TKLKQQEGAR QIARKEWKEI GKIKEIKEGY LSLVIKEISK MVIKYNAIIA | 960 | |
| | MEDLSYGFKK GRFKVERQVY QKFETMLINK LNYLVFKDIS ITENGGLLKG YQLTYIPDKL | 1020 | |
| | KNVGHQCGCI FYVPAAYTSK IDPTTGFVNI FKFKDLTVDA KREFTKKFDS IRYDSEKNLF | 1080 | |
| | CFTFDYNNFI TQNTVMSKSS WSVYTYGVRI KRRFVNGRFS NESDTIDITK DMEKTLEMTD | 1140 | |
| | INWRDGHDLR QDIIDYEIVQ HIFEIFRLTV QMRNSLSELE DRDYDRLISP VLNENNIFYD | 1200 | |
| | SAKAGDALPK DADANGAYCI ALKGLYEIKQ ITENWKEDGK FSRDKLKISN KDWFDFIQNK | 1260 | |
| | RYL | 1263 | |
| Scaffold | GTTAAGTTAT ATAGAATAAT TTCTACTGTT GTAGA | 35 | 117 |
| sequence for | | | |
| guide nucleic | | | |
| acid | | | |
| Scaffold | CTCTACAACT GATAAAGAAT TTCTACTTTT GTAGAT | 36 | 118 |
| sequence for | | | |
| guide nucleic | | | |
| acid | | | |
| Scaffold | GTCTGGCCCC AAATTTTAAT TTCTACTGTT GTAGAT | 36 | 119 |
| sequence for | | | |
| guide nucleic | | | |
| acid | | | |

Thus, one aspect of the present application provides a construct comprising one or more exogenous polynucleotides for targeted genome insertion utilizing the MAD7 endonuclease. In one embodiment, the construct further comprises a pair of homologous arms specific to a desired insertion site, and the method of targeted insertion comprises introducing the construct to cells to enable site specific homologous recombination by the cell host enzymatic machinery. In another embodiment, the method of targeted insertion in a cell comprises introducing a construct comprising one or more exogenous polynucleotides to the cell, and introducing a CRISPR MAD7 expression cassette comprising a DNA-binding domain specific to a desired insertion site to the cell. Specifically, in accordance with this disclosure, the method of targeted insertion in a cell comprises introducing a construct comprising one or more exogenous polynucleotides to the cell for insertion into particular loci in an iPSC, by introducing a MAD7 nuclease, and a gRNA comprising a guide sequence specific to a desired insertion site to the cell to enable a MAD7 mediated insertion.

In general, a guide nucleic acid can complex with a compatible nucleic acid-guided nuclease and can hybridize with a target sequence, thereby directing the nuclease to the target sequence. A guide nucleic acid can be DNA. A guide nucleic acid can be RNA. A guide nucleic acid can comprise both DNA and RNA. A guide nucleic acid can comprise modified or non-naturally occurring nucleotides. In cases where the guide nucleic acid comprises RNA, the RNA guide nucleic acid can be encoded by a DNA sequence on a polynucleotide molecule such as a plasmid, linear construct, or editing cassette as disclosed herein. In particular, in certain embodiments of the present disclosure, the guide sequence is for use with a MAD7/gRNA ribonucleoprotein (RNP) complex for insertion of a transgene into the particular loci of an iPSC, comprising: (I) a guide RNA (gRNA) polynucleotide sequence specific for the MAD7 nuclease, wherein the polynucleotide sequence comprises a guide sequence capable of hybridizing to a safe harbor locus (e.g., AAVS1, B2M, CIITA, NKG2A, TRAC, CD70, CD38, CD33, or CLYBL loci) in an iPSC, wherein when associated with MAD7 nuclease, the guide sequence directs sequence-specific binding of the MAD7 complex to the target sequence, (II) a MAD7 enzyme protein, and (III) a transgene vector comprising: (1) left and right polynucleotide sequences that are homologous to the left and right arms of the target sequence of the safe harbor locus (e.g., AAVS1, B2M, CIITA, NKG2A, TRAC, CD70, CD38, CD33, or CLYBL loci), (2) a promoter which is operably linked to (3) a polynucleotide encoding the transgene of interest, and (4) a transcription terminator sequence. In one embodiment, the guide sequence comprises a nucleotide sequence selected from SEQ ID NOs: 120-130.

Sites for targeted insertion include, but are not limited to, genomic safe harbors, which are intragenic or extragenic regions of the human genome that, theoretically, are able to accommodate predictable expression of newly inserted DNA without adverse effects on the host cell or organism. In certain embodiments, the genome safe harbor for the targeted insertion is one or more loci of genes selected from the group consisting of the AAVS1, B2M, CIITA, NKG2A, TRAC, CD70, CD38, CD33 or CLYBL loci genes.

In other embodiments, the site for targeted insertion is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for targeted deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p21), or deletion or reducing the expression level of one or more MHC class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are inserted at one or more loci of genes selected from the group consisting of B2M, TAP 1, TAP 2, Tapasin, RFXANK, CIITA, RFX5 and RFXAP genes to thereby delete or reduce the expression of the gene(s) with the insertion.

In certain embodiments, the exogenous polynucleotides are inserted at one or more loci on the chromosome of the cell, preferably the one or more loci are of genes selected from the group consisting of AAVS1, CCR5, ROSA26, collagen, HTRP, H11, GAPDH, RUNX1, B2M, TAPI, TAP2, Tapasin, NLRC5, CIITA, RFXANK, CIITA, RFX5, RFXAP, TCR a or b constant region, NKG2A, NKG2D, CD38, CIS, CBL-B, SOCS2, PD1, CTLA4, LAG3, TIM3, CD70, CD38, CD33, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. Preferably, the one or more exogenous polynucleotides are inserted at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, collagen, HTRP, H11, GAPDH, TCR and RUNX1 genes. More preferably, the one or more of the exogenous polynucleotides are inserted at the loci of CIITA, AAVS1 and B2M genes.

In certain embodiments, multiple transgenes can be inserted at sites targeted for deletion of complex (MHC) class I and MHC class II proteins. For instance, (a) a first exogenous polynucleotide may be inserted at a locus of AAVS1 gene; (b) a second exogenous polypeptide may be inserted at a locus of CIITA gene; and a third exogenous polypeptide may be inserted at a locus of B2M gene; wherein insertions of the exogenous polynucleotides delete or reduce expression of CIITA and B2M genes.

In certain embodiments, the guide RNA for insertion into the AAVS1 locus comprises a guide sequence of SEQ ID NO: 120 or a variant thereof, the left homology arm comprises the nucleotide sequence of SEQ ID NO: 60 or a fragment thereof, and the right homology arm comprises the nucleotide sequence of SEQ ID NO: 61 or a fragment thereof.

In certain embodiments, the guide RNA for insertion into the B2M locus comprises a guide sequence of SEQ ID NO: 121 or a variant thereof, the left homology arm comprises the nucleotide sequence of SEQ ID NO: 63 or a fragment thereof, and the right homology arm comprises the nucleotide sequence of SEQ ID NO: 64 or a fragment thereof.

In certain embodiments, the guide RNA for insertion into the CIITA locus comprises a guide sequence of SEQ ID NO: 122 or a variant thereof, the left homology arm comprises the nucleotide sequence of SEQ ID NO: 66 or a fragment thereof and the right homology arm comprises the nucleotide sequence of SEQ ID NO: 67 or a fragment thereof. In certain embodiments, the guide RNA for insertion into the CIITA locus comprises a guide sequence of SEQ ID NO: 126 or a variant thereof, the left homology arm comprises the nucleotide sequence of SEQ ID NO: 106 or a fragment thereof and the right homology arm comprises the nucleotide sequence of SEQ ID NO: 107 or a fragment thereof.

In certain embodiments, the guide RNA for insertion into the NKG2A locus comprises a guide sequence of SEQ ID NO: 123 or a variant thereof, the left homology arm comprises the nucleotide sequence of SEQ ID NO: 69 or a fragment thereof and the right homology arm comprises the nucleotide sequence of SEQ ID NO: 70 or a fragment thereof.

In certain embodiments, the guide RNA for insertion into the TRAC locus comprises a guide sequence of SEQ ID NO: 124 or a variant thereof, the left homology arm comprises the nucleotide sequence of SEQ ID NO: 72 or a fragment thereof and the right homology sequence arm comprises the nucleotide sequence of SEQ ID NO: 73 or a fragment thereof.

In certain embodiments, the guide RNA for insertion into the CLYBL locus comprises a guide sequence of SEQ ID NO: 125 or a variant thereof, the left homology arm comprises the nucleotide sequence of SEQ ID NO: 75 or a fragment thereof and the right homology sequence is selected from SEQ ID NO: 76 or a fragment thereof.

In certain embodiments, the guide RNA for insertion into the CD70 locus comprises a guide sequence of SEQ ID NO: 127 or a variant thereof, the left homology arm comprises the nucleotide sequence of SEQ ID NO: 109 or a fragment thereof and the right homology sequence is selected from SEQ ID NO: 110 or a fragment thereof.

In certain embodiments, the guide RNA for insertion into the CD38 locus comprises a guide sequence of SEQ ID NO: 128 or a variant thereof.

In certain embodiments, the guide RNA for insertion into the CD33 locus comprises a guide sequence of SEQ ID NO: 129 or 130 or a variant thereof.

Provided in Table 2 are targeting domain sequences for gRNA molecules (both RNA and DNA sequences are provided) and the corresponding homology arm sequences for use in the compositions and methods of the present disclosure, for example, in altering expression of or altering an iPSC target gene.

TABLE 2

| | | | | Left Homology | Right Homology |
|---|---|---|---|---|---|
| | Genomic | Guide RNA | SEQ ID | SEQ ID NO: | SEQ ID NO: |
| Target | Location | Targeting Domain Sequence | NO: | Arm | Arm |
| AAVS1 | Chr19: | UUUAUCUGUCCCCUCCACCCCACA | 120 | 60 | 61 |
| | 55115778 | TTTATCTGTCCCCTCCACCCCACA | 59 | | |
| B2M | Chr15: | UUUACUCACGUCAUCCAGCAGAGA | 121 | 63 | 64 |
| | 44715462 | TTTACTCACGTCATCCAGCAGAGA | 62 | | |
| CIITA | Chr:16 | UUUACCUUGGGGCUCUGACAGGUA | 122 | 66 | 67 |
| | 10877367 | TTTACCTTGGGGCTCTGACAGGTA | 65 | | |
| CLYBL | Chr:13 | AGAGUGAUCACAGCUCUGACUAAA | 123 | 69 | 70 |
| | 99822675 | AGAGTGATCACAGCTCTGACTAAA | 68 | | |
| NKG2A | Chr:12 | CUCAGACCUGAAUCUGCCCCCAAA | 124 | 72 | 73 |
| | 10451131 | CTCAGACCTGAATCTGCCCCCAAA | 71 | | |
| TRAC | Chr:14 | GUGUACCAGCUGAGAGACUCUAAA | 125 | 75 | 76 |
| | 22547532 | GTGTACCAGCTGAGAGACTCTAAA | 74 | | |
| CIITA | — | UUUCUGCCCAACUUCUGCUGGCAU | 126 | 106 | 107 |
| Exon 5 | | TTTCTGCCCAACTTCTGCTGGCAT | 105 | | |
| CD70 | — | UUUGGUCCCAUUGGUCGCGGGCUU | 127 | 109 | 110 |
| Exon 1 | | TTTGGTCCCATTGGTCGCGGGCTT | 108 | | |
| CD38 | — | UUUCCCGAGACCGUCCUGGCGCG | 128 | — | — |
| Exon 1 | | TTTCCCGAGACCGTCCTGGCGCG | 111 | | |
| CD33 | Chr:19 | UUUGUCUGCAGGGAAACAAGAGACC | 129 | — | — |
| Exon 5 | 51235170 | TTTGTCTGCAGGGAAACAAGAGACC | 112 | | |
| CD33 | Chr:19 | UUUGGAGUGGCCGGGUUCUAGAGUG | 130 | — | — |
| Exon 3 | 51225838 | TTTGGAGTGGCCGGGTTCTAGAGTG | 113 | | |

Homology Arms

Whether single-stranded or double-stranded, donor templates generally include one or more regions that are homologous to regions of DNA, e.g., a target nucleic acid, within or near (e.g., flanking or adjoining) a target sequence to be cleaved, e.g., the cleavage site. These homologous regions are referred to here as “homology arms,” and are illustrated schematically below:
[5′ homology arm]-[replacement sequence]-[3′ homology arm].

The homology arms of the donor templates described herein may be of any suitable length, provided such length is sufficient to allow efficient resolution of a cleavage site on a targeted nucleic acid by a DNA repair process requiring a donor template. In certain embodiments, where amplification by, e.g., PCR, of the homology arm is desired, the homology arm is of a length such that the amplification may be performed. In certain embodiments, where sequencing of the homology arm is desired, the homology arm is of a length such that the sequencing may be performed. In certain embodiments, where quantitative assessment of amplicons is desired, the homology arms are of such a length such that a similar number of amplifications of each amplicon is achieved, e.g., by having similar G/C content, amplification temperatures, etc. In certain embodiments, the homology arm is double-stranded. In certain embodiments, the double stranded homology arm is single stranded.

In certain embodiments, the 5′ homology arm is between 50 to 250 nucleotides in length. In certain embodiments, the 5′ homology arm is about 50 nucleotides, about 75 nucleotides, about 100 nucleotides, about 125 nucleotides, about 150 nucleotides, about 175 nucleotides, about 200 nucleotides, about 225 nucleotides, or about 250 nucleotides in length.

In certain embodiments, the 3′ homology arm is between 50 to 250 nucleotides in length. In certain embodiments, the 3′ homology arm is about 50 nucleotides, about 75 nucleotides, about 100 nucleotides, about 125 nucleotides, about 150 nucleotides, about 175 nucleotides, about 200 nucleotides, about 225 nucleotides, or about 250 nucleotides in length.

The 5′ and 3′ homology arms can be of the same length or can differ in length. In certain embodiments, the 5′ and 3′ homology arms are amplified to allow for the quantitative assessment of gene editing events, such as targeted insertion, at a target nucleic acid. In certain embodiments, the quantitative assessment of the gene editing events may rely on the amplification of both the 5′ junction and 3′ junction at the site of targeted insertion by amplifying the whole or a part of the homology arm using a single pair of PCR primers in a single amplification reaction. Accordingly, although the length of the 5′ and 3′ homology arms may differ, the length of each homology arm should be capable of amplification (e.g., using PCR), as desired. Moreover, when amplification of both the 5′ and the difference in lengths of the 5′ and 3′ homology arms in a single PCR reaction is desired, the length difference between the 5′ and 3′ homology arms should allow for PCR amplification using a single pair of PCR primers.

### IV. Transgenes for Insertion in iPSCs

According to embodiments of the application, an iPSC is engineered by the insertion of one or more transgenes using the described MAD7/gRNA ribonucleoprotein (RNP) complex of this disclosure. A host of different transgenes comprising a gene of interest may be inserted utilizing the RNP complex, guide sequences and homology arms in accordance with this disclosure. Exemplary transgenes are further discussed below:
A. Chimeric Antigen Receptors (“CARs”)

At least one of the transgenes that may be inserted is one encoding an exogenous chimeric antigen receptor (CAR), such as a CAR targeting a tumor antigen.

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen binding domain and/or an antigen binding fragment. The antigen binding fragment can, for example, be an antibody or antigen binding fragment thereof that specifically binds a tumor antigen. The antigen binding fragments of the application possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a VHH having binding specificity for a first epitope, and a VHH having binding specificity for a second epitope.

As used herein, an antigen binding domain or antigen binding fragment that “specifically binds to a tumor antigen” refers to an antigen binding domain or antigen binding fragment that binds a tumor antigen, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

The smaller the value of the KD of an antigen binding domain or antigen binding fragment, the higher affinity that the antigen binding domain or antigen binding fragment binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is a Fab fragment, a Fab′ fragment, a F(ab′)2 fragment, a scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment. In some embodiments, the antigen-binding fragment is a VHH.

In some embodiments, at least one of the extracellular tag-binding domain, the antigen-binding domain, or the tag comprises a single-domain antibody or nanobody.

In some embodiments, at least one of the extracellular tag-binding domain, the antigen-binding domain, or the tag comprises a VHH.

In some embodiments, the extracellular tag-binding domain and the tag each comprise a VHH.

In some embodiments, the extracellular tag-binding domain, the tag, and the antigen-binding domain each comprise a VHH.

In some embodiments, at least one of the extracellular tag-binding domain, the antigen-binding domain, or the tag comprises an scFv.

In some embodiments, the extracellular tag-binding domain and the tag each comprise an scFv.

In some embodiments, the extracellular tag-binding domain, the tag, and the antigen-binding domain each comprise a scFv.

Alternative scaffolds to immunoglobulin domains that exhibit similar functional characteristics, such as high-affinity and specific binding of target biomolecules, may also be used in the CARs of the present disclosure. Such scaffolds have been shown to yield molecules with improved characteristics, such as greater stability or reduced immunogenicity. Non-limiting examples of alternative scaffolds that may be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™ or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD₃ (e.g., Tetranectin); thioredoxin (e.g., peptide aptamer); KALBITOR®; the β-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biolechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety for all intended purposes).

In some embodiments, the alternative scaffold is Affilin or Centyrin.

In some embodiments, the first polypeptide of the CARs of the present disclosure comprises a leader sequence. The leader sequence may be positioned at the N-terminus the extracellular tag-binding domain. The leader sequence may be optionally cleaved from the extracellular tag-binding domain during cellular processing and localization of the CAR to the cellular membrane. Any of various leader sequences known to one of skill in the art may be used as the leader sequence. Non-limiting examples of peptides from which the leader sequence may be derived include granulocyte-macrophage colony-stimulating factor receptor (GMCSFR), FcεR, human immunoglobulin (IgG) heavy chain (HC) variable region, CD8a, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1.

In some embodiments, the first polypeptide of the CARs of the present disclosure comprise a transmembrane domain, fused in frame between the extracellular tag-binding domain and the cytoplasmic domain.

The transmembrane domain may be derived from the protein contributing to the extracellular tag-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, R or (chain of the T cell receptor (TCR), CD28, CD3 epsilon, CD45, CD4, CD5, CD8, CD8a, CD9, CD16, CD22, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η, or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η, or FcεR1γ and −β, MIB1 (Igα.), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the CAR of the present disclosure comprises a spacer region between the extracellular tag-binding domain and the transmembrane domain, wherein the tag-binding domain, linker, and the transmembrane domain are in frame with each other.

The term “spacer region” as used herein generally means any oligo- or polypeptide that functions to link the tag-binding domain to the transmembrane domain. A spacer region can be used to provide more flexibility and accessibility for the tag-binding domain. A spacer region may comprise up to 300 amino acids, preferably 10 to 100 amino acids and most preferably 25 to 50 amino acids. A spacer region may be derived from all or part of naturally occurring molecules, such as from all or part of the extracellular region of CD8, CD4 or CD28, or from all or part of an antibody constant region. Alternatively, the spacer region may be a synthetic sequence that corresponds to a naturally occurring spacer region sequence, or may be an entirely synthetic spacer region sequence. Non-limiting examples of spacer regions which may be used in accordance to the disclosure include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer may be mutated to prevent Fc receptor binding.

In some embodiments, the spacer region comprises a hinge domain. The hinge domain may be derived from CD8a, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 55) from IgG1, ERKCCVECPPCP (SEQ ID NO: 56) from IgG2, ELKTPLGDTTHTCPRCP(EPKSCDTPPPCPRCP)₃ (SEQ ID NO: 57) from IgG3, and ESKYGPPCPSCP (SEQ ID NO: 58) from IgG4 (see also Wypych et al., JBC 2008 283(23): 16194-16205, which is incorporated herein by reference in its entirety for all purposes). In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

TABLE 3
Hinge Sequences

| | SEQ ID |
|---|---|
| Sequence | NO |
| EPKSCDKTHTCPPCP | 55 |
| ERKCCVECPPCP | 56 |
| ELKTPLGDTTHTCPRCPEPKSCDTPPPCPRCPEPKSC | 57 |
| DTPPPCPRCPEPKSCDTPPPCPRCP | |
| ESKYGPPCPSCP | 58 |

In certain aspects, the first polypeptide of CARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, CD66d, CD79A, and CD79B.

In some embodiments, the cytoplasmic domain comprises a CD3ζ signaling domain. In one embodiment, the CD3ζ signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10.

In one embodiment, the co-stimulatory signaling domain is derived from OX40. In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15.

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In one embodiment, the co-stimulatory signaling domain is derived from CD28. In one embodiment, the CD28 co-stimulatory signaling domain comprises the amino acid sequence set forth in SEQ ID NO: 20, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 20.

In one embodiment, the CAR of the present disclosure comprises a hinge region, a transmembrane region and a co-stimulatory signaling domain all derived from CD28. In one embodiment, the hinge region, transmembrane region and co-stimulatory signaling domain derived from CD28 comprises the amino acid sequence set forth in SEQ ID NO: 5, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 5.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 4.

TABLE 4

| | | | |
|---|---|---|---|
| | | UniProt | SEQ ID |
| CAR regions | Sequence | Id | NO |

CD19 CAR:

| | | | |
|---|---|---|---|
| GMCSFR | MLLLVTSLLLCELPHPAFLLIP | | 1 |
| Signal Peptide | | | |
| FMC63 VH | EVKLQESGPGLVAPSQSLSVTCTVSGVSLPDYGVSW | | 2 |
| | IRQPPRKGLEWLGVIWGSETTYYNSALKSRLTIIKDN | | |
| | SKSQVFLKMNSLQTDDTAIYYCAKHYYYGGSYAM | | |
| | DYWGQGTSVTVSS | | |
| Whitlow | GSTSGSGKPGSGEGSTKG | | 3 |
| Linker | | | |
| FMC63 VL | DIQMTQTTSSLSASLGDRVTISCRASQDISKYLNWY | | 4 |
| | QQKPDGTVKLLIYHTSRLHSGVPSRFSGSGSGTDYS | | |
| | LTISNLEQEDIATYFCQQGNTLPYTFGGGTKLEIT | | |
| CD28 | IEVMYPPPYLDNEKSNGTIIHVKGKHLCPSPLFPGPS | P10747-1 | 5 |
| (AA 114-220) | KPFWVLVVVGGVLACYSLLVTVAFIIFWVRSKRSR | | |
| | LLHSDYMNMTPRRPGPTRKHYQPYAPPRDFAAYRS | | |
| CD3-zeta | RVKFSRSADAPAYQQGQNQLYNELNLGRREEYDV | P20963-3 | 6 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| FMC63 scFV | EVKLQESGPGLVAPSQSLSVTCTVSGVSLPDYGVS | | 7 |
| | WIRQPPRKGLEWLGVIWGSETTYYNSALKSRLTIIK | | |
| | DNSKSQVFLKMNSLQTDDTAIYYCAKHYYYGGSY | | |
| | AMDYWGQGTSVTVSSGSTSGSGKPGSGEGSTKGDI | | |
| | QMTQTTSSLSASLGDRVTISCRASQDISKYLNWYQQ | | |
| | KPDGTVKLLIYHTSRLHSGVPSRFSGSGSGTDYSLTI | | |
| | SNLEQEDIATYFCQQGNTLPYTFGGGTKLEIT | | |

Signaling Domains:

| | | | |
|---|---|---|---|
| 41BB | KRGRKKLLYIFKQPFMRPVQTTQEEDGCSCRFPEEE | Q07011 | 8 |
| (AA 214-255) | EGGCEL | | |
| IL2Rb | NCRNTGPWLKKVLKCNTPDPSKFFSQLSSEHGGDV | P14784 | 9 |
| (AA 266-551) | QKWLSSPFPSSSFSPGGLAPEISPLEVLERDKVTQLL | | |
| | PLNTDAYLSLQELQGQDPTHLV | | |
| CD40 | KKVAKKPTNKAPHPKQEPQEINFPDDLPGSNTAAPV | P25942 | 10 |
| (AA 216-277) | QETLHGCQPVTQEDGKESRISVQERQ | | |
| OX40 | ALYLLRRDQRLPPDAHKPPGGGSFRTPIQEEQADAH | P43489 | 11 |
| (AA 236-277) | STLAKI | | |
| CD80 | TYCFAPRCRERRRNERLRRESVRPV | P33681 | 12 |
| (AA 264-288) | | | |
| CD86 | KWKKKKRPRNSYKCGTNTMEREESEQTKKREKIHI | P42081 | 13 |
| (AA269-329) | PERSDEAQRVFKSSKTSSCDKSDTCF | | |
| CD27 | QRRKYRSNKGESPVEPAEPCHYSCPREEEGSTIPIQE | P26842 | 14 |
| (AA 213-260) | DYRKPEPACSP | | |
| ICOS | CWLTKKKYSSSVHDPNGEYMFMRAVNTAKKSRLT | Q9Y6W8 | 15 |
| (AA 162-199) | DVTL | | |
| NKG2D | MGWIRGRRSRHSWEMSEFHNYNLDLKKSDF | P26718 | 16 |
| (AA 1-51) | STRWQKQRCPVVKSKCRENAS | | |
| DAP 10 | LCARPRRSPAQEDGKVYINMPGRG | Q9UBK5 | 17 |
| (AA 70-93) | | | |
| DAP 12 | YFLGRLVPRGRGAAEAATRKQRITETESPYQELQGQ | O54885 | 18 |
| (AA 62-113) | RSDVYSDLNTQRPYYK | | |
| 2B4/CD244 | WRRKRKEKQSETSPKEFLTIYEDVKDLKTRRNHEQ | Q9BZW8 | 19 |
| (AA 251-370) | EQTFPGGGSTIYSMIQSQSSAPTSQEPAYTLYSLIQPS | | |
| | RKSGSRKRNHSPSFNSTIYEVIGKSQPKAQNPARLSR | | |
| | KELENFDVYS | | |
| CD3-zeta | RVKFSRSADAPAYQQGQNQLYNELNLGRREEYDV | P20963-3 | 6 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| CD28 | RSKRSRLLHSDYMNMTPRRPGPTRKHYQPYAPPRD | P10747-1 | 20 |
| (AA 180-220) | FAAYRS | | |

Spacer/Hinge:

| | | | |
|---|---|---|---|
| CD8 | TTTPAPRPPTPAPTIASQPLSLRPEACRPAAGGAVHT | P01732 | 21 |
| (AA 136-182) | RGLDFACDIY | | |
| CD28 | IEVMYPPPYLDNEKSNGTIIHVKGKHLCPSPLFPGPS | P10747-1 | 22 |
| (AA 114-151) | KP | | |

Transmembrane:

| | | | |
|---|---|---|---|
| CD8 | IYIWAPLAGTCGVLLLSLVIT | P01732 | 23 |
| (AA 183-203) | | | |
| CD28 | FWVLVVVGGVLACYSLLVTVAFIIFWV | P10747-1 | 24 |
| (AA 153-179) | | | |

Linkers:

| | | |
|---|---|---|
| Whitlow | GSTSGSGKPGSGEGSTKG | 3 |
| Linker | | |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 5 | GGGSGGGSGGGS | 28 |
| Linker 6 | GGGSGGGSGGGSGGGS | 29 |
| Linker 7 | GGGSGGGSGGGSGGGSGGGS | 30 |
| Linker 8 | GGGGSGGGGSGGGGSGGGGS | 31 |
| Linker 9 | GGGGSGGGGSGGGGSGGGGSGGGGS | 32 |
| Linker 10 | IRPRAIGGSKPRVA | 33 |
| Linker 11 | GKGGSGKGGSGKGGS | 34 |
| Linker 12 | GGKGSGGKGSGGKGS | 35 |
| Linker 13 | GGGKSGGGKSGGGKS | 36 |
| Linker 14 | GKGKSGKGKSGKGKS | 37 |
| Linker 15 | GGGKSGGKGSGKGGS | 38 |
| Linker 16 | GKPGSGKPGSGKPGS | 39 |
| Linker 17 | GKPGSGKPGSGKPGSGKPGS | 40 |
| Linker 18 | GKGKSGKGKSGKGKSGKGKS | 41 |
| Linker 19 | STAGDTHLGGEDFD | 42 |
| Linker 20 | GEGGSGEGGSGEGGS | 43 |
| Linker 21 | GGEGSGGEGSGGEGS | 44 |
| Linker 22 | GEGESGEGESGEGES | 45 |
| Linker 23 | GGGESGGEGSGEGGS | 46 |
| Linker 24 | GEGESGEGESGEGESGEGES | 47 |
| Linker 25 | PRGASKSGSASQTGSAPGS | 48 |
| Linker 26 | GTAAAGAGAAGGAAAGAAG | 49 |
| Linker 27 | GTSGSSGSGSGGSGSGGGG | 50 |
| Linker 28 | GSGS | 51 |
| Linker 29 | APAPAPAPAP | 52 |
| Linker 30 | APAPAPAPAPAPAPAPAPAP | 53 |
| Linker 31 | AEAAAKEAAAKEAAAAKEAAAAKEAAAAKAAA | 54 |

In some embodiments, the antigen-binding domain of the second polypeptide binds to an antigen. The antigen-binding domain of the second polypeptide may bind to more than one antigen or more than one epitope in an antigen. For example, the antigen-binding domain of the second polypeptide may bind to two, three, four, five, six, seven, eight or more antigens. As another example, the antigen-binding domain of the second polypeptide may bind to two, three, four, five, six, seven, eight or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79, BCMA, GPRC5D, SLAM F7, CD33, CLL1, CD123, and CD70. Non-limiting examples of tumor antigens associated with bladder cancer include Nectin-4 and SLITRK6.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 2, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In one embodiment, the anti-CD19 scFv comprises a light chain variable region (VL) comprising the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In one embodiment, the anti-CD19 scFv comprises the amino acid sequence set forth in SEQ ID NO: 7, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 7.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, fillaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elogation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB′/B), U1RNP, A2/B1 hnRNP, Ro (SSA), and La (SSB) antigens.

In various embodiments, the scFv fragment used in the CAR of the present disclosure may include a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G4S)₃ linker. In one embodiment, the (G4S)₃ linker comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences may include portions of immunoglobulin hinge area, CL or CH1 derived from any immunoglobulin heavy or light chain isotype. Exemplary linkers that may be used include any of SEQ ID NOs: 26-54 in Table 4. Additional linkers are described for example in Int. Pat. Publ. No. WO2019/060695, incorporated by reference herein in its entirety for all intended purposes.
B. Artificial cell death polypeptides

Another potential transgene for insertion in accordance with this disclosure is an exogenous polynucleotide encoding an artificial cell death polypeptide.

As used herein, the term “an artificial cell death polypeptide” refers to an engineered protein designed to prevent potential toxicity or otherwise adverse effects of a cell therapy. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g. an antibody, that when activated, triggers apoptosis and/or cell death of a therapeutic cell. In certain embodiments, the mechanism of action of the artificial cell death polypeptide is metabolic, dimerization-inducing or therapeutic monoclonal antibody mediated.

In certain embodiments, artificial cell death polypeptide is an inactivated cell surface receptor that comprises an epitope specifically recognized by an antibody, particularly a monoclonal antibody, which is also referred to herein as a monoclonal antibody-specific epitope. When expressed by iPSCs or derivative cells thereof, the inactivated cell surface receptor is signaling inactive or significantly impaired, but can still be specifically recognized by an antibody. The specific binding of the antibody to the inactivated cell surface receptor enables the elimination of the iPSCs or derivative cells thereof by ADCC and/or ADCP mechanisms, as well as, direct killing with antibody drug conjugates with toxins or radionuclides.

In certain embodiments, the inactivated cell surface receptor comprises an epitope that is selected from epitopes specifically recognized by an antibody, including but not limited to, ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, or ustekinumab.

Epidermal growth factor receptor, also known as EGFR, ErbB1 and HER1, is a cell-surface receptor for members of the epidermal growth factor family of extracellular ligands. As used herein, “truncated EGFR,” “tEGFR,” “short EGFR” or “sEGFR” refers to an inactive EGFR variant that lacks the EGF-binding domains and the intracellular signaling domains of the EGFR. An exemplary tEGFR variant contains residues 322-333 of domain 2, all of domains 3 and 4 and the transmembrane domain of the native EGFR sequence containing the cetuximab binding epitope. Expression of the tEGFR variant on the cell surface enables cell elimination by an antibody that specifically binds to the tEGFR, such as cetuximab (Erbitux®), as needed. Due to the absence of the EGF-binding domains and intracellular signaling domains, tEGFR is inactive when expressed by iPSCs or derivative cell thereof.

An exemplary inactivated cell surface receptor of the application comprises a tEGFR variant. In certain embodiments, expression of the inactivated cell surface receptor in an engineered immune cell expressing a chimeric antigen receptor (CAR) induces cell suicide of the engineered immune cell when the cell is contacted with an anti-EGFR antibody. Methods of using inactivated cell surface receptors are described in WO2019/070856, WO2019/023396, WO2018/058002, the disclosure of which is incorporated herein by reference. For example, a subject who has previously received an engineered immune cell of the present disclosure that comprises a heterologous polynucleotide encoding an inactivated cell surface receptor comprising a tEGFR variant can be administered an anti-EGFR antibody in an amount effective to ablate in the subject the previously administered engineered immune cell.

In certain embodiments, the anti-EGFR antibody is cetuximab, matuzumab, necitumumab or panitumumab, preferably the anti-EGFR antibody is cetuximab.

In certain embodiments, the tEGFR variant comprises or consists of an amino acid sequence at least 90%, such as at least 90%, 91%, 92%, 93%, 94%, 95%, 96%, 97%, 98%, 99% or 100%, identical to SEQ ID NO: 77, preferably the amino acid sequence of SEQ ID NO: 77.

In some embodiments, the inactivated cell surface receptor comprises one or more epitopes of CD79b, such as an epitope specifically recognized by polatuzumab vedotin. In certain embodiments, the CD79b epitope comprises or consists of an amino acid sequence at least 90%, such as at least 90%, 91%, 92%, 93% 94%, 95%, 96%, 97% 98%, 99% or 100%, identical to SEQ ID NO: 81, preferably the amino acid sequence of SEQ ID NO: 81.

In some embodiments, the inactivated cell surface receptor comprises one or more epitopes of CD20, such as an epitope specifically recognized by rituximab. In certain embodiments, the CD20 epitope comprises or consists of an amino acid sequence at least 90%, such as at least 90%, 91%, 92%, 93%, 94%, 95%, 96%, 97%, 98%, 99% or 100%, identical to SEQ ID NO: 82, preferably the amino acid sequence of SEQ ID NO: 82.

In some embodiments, the inactivated cell surface receptor comprises one or more epitopes of Her 2 receptor or ErbB, such as an epitope specifically recognized by trastuzumab. In certain embodiments, the monoclonal antibody-specific epitope comprises or consists of an amino acid sequence at least 90%, such as at least 90%, 91%, 92%, 93%, 94%, 95%, 96%, 97%, 98%, 99% or 100%, identical to SEQ ID NO: 84, preferably the amino acid sequence of SEQ ID NO: 84.

In some embodiments, the genome-engineered iPSCs generated using the above method comprise one or more different exogenous polynucleotides encoding proteins comprising caspase, thymidine kinase, cytosine deaminase, B-cell CD20, ErbB2 or CD79b wherein when the genome-engineered iPSCs comprise two or more suicide genes, the suicide genes are integrated in different safe harbor locus such as AAVS1, B2M, CIITA, NKG2A, TRAC, CD70, CD38, CD33, or CLYBL.
C. Cytokines

In some embodiments the transgene for insertion is one encoding a cytokine, such as interleukin-15 or interleukin-2.

As used herein “Interleukin-15” or “IL-15” refers to a cytokine that regulates T and NK cell activation and proliferation, or a functional portion thereof. A “functional portion” (“biologically active portion”) of a cytokine refers to a portion of the cytokine that retains one or more functions of full length or mature cytokine. Such functions for IL-15 include the promotion of NK cell survival, regulation of NK cell and T cell activation and proliferation as well as the support of NK cell development from hematopoietic stem cells. As will be appreciated by those of skill in the art, the sequence of a variety of IL-15 molecules are known in the art. In certain embodiments, the IL-15 is a wild-type IL-15. In certain embodiments, the IL-15 is a human IL-15. In certain embodiments, the IL-15 comprises an amino acid sequence at least 90%, such as at least 90%, 91%, 92%, 93%, 94%, 95%, 96%, 97%, 98%, 99% or 100%, identical to SEQ ID NO: 79, preferably the amino acid sequence of SEQ ID NO: 79.

As used herein “Interleukin-2” refers to a cytokine that regulates T and NK cell activation and proliferation, or a functional portion thereof. In certain embodiments, the IL-2 is a wild-type IL-2. In certain embodiments, the IL-2 is a human IL-2. In certain embodiments, the IL-2 comprises an amino acid sequence at least 90%, such as at least 90%, 91%, 92%, 93%, 94%, 95%, 96%, 97%, 98%, 99% or 100%, identical to SEQ ID NO: 85, preferably the amino acid sequence of SEQ ID NO: 85.

In certain embodiments, the transgene can include an exogenous gene encoding an inactivated cell surface receptor comprising a monoclonal antibody-specific epitope operably linked to a cytokine, preferably by an autoprotease peptide sequence. Examples of the autoprotease peptide include, but are not limited to, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In one embodiment, the autoprotease peptide is an autoprotease peptide of porcine tesehovirus-1 2A (P2A). In certain embodiments, the autoprotease peptide comprises an amino acid sequence at least 90%, such as at least 90%, 91%, 92%, 93%, 94%, 95%, 96%, 97%, 98%, 99% or 100%, identical to SEQ ID NO: 78, preferably the amino acid sequence of SEQ ID NO: 78.

In certain embodiments, an inactivated cell surface receptor comprises a truncated epithelial growth factor (tEGFR) variant operably linked to an interleukin-15 (IL-15) or IL-2 by an autoprotease peptide sequence. In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 86, preferably the amino acid sequence of SEQ ID NO: 86.

In some embodiments, an inactivated cell surface receptor further comprises a signal sequence. In certain embodiments, the signal sequence comprises an amino acid sequence at least 90%, such as at least 90%, 91%, 92%, 93%, 94%, 95%, 96%, 97%, 98%, 99% or 100%, identical to SEQ ID NO: 80, preferably the amino acid sequence of SEQ ID NO: 80.

In some embodiments, an inactivated cell surface receptor further comprises a hinge domain. In some embodiments, the hinge domain is derived from CD8. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21.

In certain embodiments, an inactivated cell surface receptor further comprises a transmembrane domain. In some embodiments, the transmembrane domain is derived from CD8. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23.

In certain embodiment, an inactivated cell surface receptor comprises one or more epitopes specifically recognized by an antibody in its extracellular domain, a transmembrane region and a cytoplasmic domain. In some embodiments, the inactivated cell surface receptor further comprises a hinge region between the epitope(s) and the transmembrane region. In some embodiments, the inactivated cell surface receptor comprises more than one epitopes specifically recognized by an antibody, the epitopes can have the same or different amino acid sequences, and the epitopes can be linked together via a peptide linker, such as a flexible peptide linker have the sequence of (GGGGS)n, wherein n is an integer of 1-8 (SEQ ID NOs: 87, 101, 25, 31, 32, and 102-104, respectively). In some embodiments, the inactivated cell surface receptor further comprises a cytokine, such as an IL-15 or IL-2. In certain embodiments, the cytokine is in the cytoplasmic domain of the inactivated cell surface receptor. Preferably, the cytokine is operably linked to the epitope(s) specifically recognized by an antibody, directly or indirectly, via an autoprotease peptide sequence, such as those described herein. In some embodiments, the cytokine is indirectly linked to the epitope(s) by connecting to the transmembrane region via the autoprotease peptide sequence.

Non-limiting exemplary inactivated cell surface receptor regions and sequences are provided in Table 5.

TABLE 5

| | | |
|---|---|---|
| | | SEQ ID |
| Regions | Sequence | NO |

tEGFR-IL15:

| | | |
|---|---|---|
| tEGFR | MRPSGTAGAALLALLAALCPASRAGVRKCKKCEGPCRKVCN | 77 |
| | GIGIGEFKDSLSINATNIKHFKNCTSISGDLHILPVAFRGDSFTH | |
| | TPPLDPQELDILKTVKEITGFLLIQAWPENRTDLHAFENLEIIRG | |
| | RTKQHGQFSLAVVSLNITSLGLRSLKEISDGDVIISGNKNLCYA | |
| | NTINWKKLFGTSGQKTKIISNRGENSCKATGQVCHALCSPEG | |
| | CWGPEPRDCVSCRNVSRGRECVDKCNLLEGEPREFVENSECI | |
| | QCHPECLPQAMNITCTGRGPDNCIQCAHYIDGPHCVKTCPAG | |
| | VMGENNTLVWKYADAGHVCHLCHPNCTYGCTGPGLEGCPT | |
| | NGPKIPSIATGMVGALLLLLVVALGIGLFM | |
| P2A | ATNFSLLKQAGDVEENPGP | 78 |
| IL-15 | MRISKPHLRSISIQCYLCLLLNSHFLTEAGIHVFILGCFSAGLPK | 79 |
| | TEANWVNVISDLKKIEDLIQSMHIDATLYTESDVHPSCKVTA | |
| | MKCFLLELQVISLESGDASIHDTVENLIILANNSLSSNGNVTES | |
| | GCKECEELEEKNIKEFLQSFVHIVQMFINTS | |

CD79b-IL15:

| | | |
|---|---|---|
| Signal | MEFGLSWVFLVALFRGVQC | 80 |
| Sequence | | |
| CD79b | ARSEDRYRNPKGSACSRIWQS | 81 |
| epitope | | |
| CD8 (AA | TTTPAPRPPTPAPTIASQPLSLRPEACRPAAGGAVHTRGLDFA | 21 |
| 136-182) | CDIY | |
| CD8 (AA | IYIWAPLAGTCGVLLLSLVIT | 23 |
| 183-203) | | |
| P2A | ATNFSLLKQAGDVEENPGP | 78 |
| IL-15 | MRISKPHLRSISIQCYLCLLLNSHFLTEAGIHVFILGCFSAGLPK | 79 |
| | TEANWVNVISDLKKIEDLIQSMHIDATLYTESDVHPSCKVTA | |
| | MKCFLLELQVISLESGDASIHDTVENLIILANNSLSSNGNVTES | |
| | GCKECEELEEKNIKEFLQSFVHIVQMFINTS | |

CD20 mimitope-IL15:

| | | |
|---|---|---|
| Signal | MEFGLSWVFLVALFRGVQC | 80 |
| Sequence | | |
| CD20 | ACPYANPSLC | 82 |
| mimitope | | |
| Linker | GGGSGGGS | 83 |
| CD8 (AA | TTTPAPRPPTPAPTIASQPLSLRPEACRPAAGGAVHTRGLDFA | 21 |
| 136-182) | CDIY | |
| CD8 (AA | IYIWAPLAGTCGVLLLSLVIT | 23 |
| 183-203) | | |
| P2A | ATNFSLLKQAGDVEENPGP | 78 |
| IL-15 | MRISKPHLRSISIQCYLCLLLNSHFLTEAGIHVFILGCFSAGLPK | 79 |
| | TEANWVNVISDLKKIEDLIQSMHIDATLYTESDVHPSCKVTA | |
| | MKCFLLELQVISLESGDASIHDTVENLIILANNSLSSNGNVTES | |
| | GCKECEELEEKNIKEFLQSFVHIVQMFINTS | |

ErbB epitope-IL15:

| | | |
|---|---|---|
| Signal | MEFGLSWVFLVALFRGVQC | 80 |
| Sequence | | |
| ErbB | EGLACHQLCARGHCWGPGPTQCVNCSQFLRGQECVEECRVL | 84 |
| epitope | QGLPREYVNARHCLPCHPECQPQNGSVTCFGPEADQCVACA | |
| | HYKDPPFCVARCPSGVKPDLSYMPIWKFPDEEGACQPCPINCT | |
| | HSCVDLDDKGCPAEQRASPLTSIISAVVGILLVVVLGVVFGILI | |
| | GGGGSGG | |
| P2A | ATNFSLLKQAGDVEENPGP | 78 |
| IL-15 | MRISKPHLRSISIQCYLCLLLNSHFLTEAGIHVFILGCFSAGLPK | 79 |
| | TEANWVNVISDLKKIEDLIQSMHIDATLYTESDVHPSCKVTA | |
| | MKCFLLELQVISLESGDASIHDTVENLIILANNSLSSNGNVTES | |
| | GCKECEELEEKNIKEFLQSFVHIVQMFINTS | |

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 88, preferably the amino acid sequence of SEQ ID NO: 88.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 89, preferably the amino acid sequence of SEQ ID NO: 89.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 90, preferably the amino acid sequence of SEQ ID NO: 90.

TABLE 6

| | | SEQ ID |
|---|---|---|
| Regions | Sequence | NO |
| IL-2 | MYRMQLLSCIALSLALVTNSAPTSSSTKKTQLQLEHLLLDLQ | 85 |
| | MILNGINNYKNPKLTRMLTFKFYMPKKATELKHLQCLEEELK | |
| | PLEEVLNLAQSKNFHLRPRDLISNINVIVLELKGSETTFMCEYA | |
| | DETATIVEFLNRWITFCQSIISTLT | |
| tEGFR- | MRPSGTAGAALLALLAALCPASRAGVRKCKKCEGPCRKVCN | 86 |
| P2A-IL15 | GIGIGEFKDSLSINATNIKHFKNCTSISGDLHILPVAFRGDSFTH | |
| | TPPLDPQELDILKTVKEITGFLLIQAWPENRTDLHAFENLEIIRG | |
| | RTKQHGQFSLAVVSLNITSLGLRSLKEISDGDVIISGNKNLCYA | |
| | NTINWKKLFGTSGQKTKIISNRGENSCKATGQVCHALCSPEGC | |
| | WGPEPRDCVSCRNVSRGRECVDKCNLLEGEPREFVENSECIQ | |
| | CHPECLPQAMNITCTGRGPDNCIQCAHYIDGPHCVKTCPAGV | |
| | MGENNTLVWKYADAGHVCHLCHPNCTYGCTGPGLEGCPTN | |
| | GPKIPSIATGMVGALLLLLVVALGIGLFMSGSGATNFSLLKQA | |
| | GDVEENPGPMRISKPHLRSISIQCYLCLLLNSHFLTEAGIHVFIL | |
| | GCFSAGLPKTEANWVNVISDLKKIEDLIQSMHIDATLYTESDV | |
| | HPSCKVTAMKCFLLELQVISLESGDASIHDTVENLIILANNSLS | |
| | SNGNVTESGCKECEELEEKNIKEFLQSFVHIVQMFINTS | |
| (G4S)1 | GGGGS | 87 |
| Linker | | |
| CD79b- | MEFGLSWVFLVALFRGVQCARSEDRYRNPKGSACSRIWQSTT | 88 |
| P2A-IL15 | TPAPRPPTPAPTIASQPLSLRPEACRPAAGGAVHTRGLDFACDI | |
| | YIWAPLAGTCGVLLLSLVITATNFSLLKQAGDVEENPGPMRIS | |
| | KPHLRSISIQCYLCLLLNSHFLTEAGIHVFILGCFSAGLPKTEAN | |
| | WVNVISDLKKIEDLIQSMHIDATLYTESDVHPSCKVTAMKCFL | |
| | LELQVISLESGDASIHDTVENLIILANNSLSSNGNVTESGCKEC | |
| | EELEEKNIKEFLQSFVHIVQMFINTS | |
| CD20 | MEFGLSWVFLVALFRGVQCACPYANPSLCGGGGSGGGGSAC | 89 |
| Mimitope- | PYANPSLCTTTPAPRPPTPAPTIASQPLSLRPEACRPAAGGAVH | |
| P2A-IL15 | TRGLDFACDIYIWAPLAGTCGVLLLSLVITATNFSLLKQAGDV | |
| | EENPGPMRISKPHLRSISIQCYLCLLLNSHFLTEAGIHVFILGCF | |
| | SAGLPKTEANWVNVISDLKKIEDLIQSMHIDATLYTESDVHPS | |
| | CKVTAMKCFLLELQVISLESGDASIHDTVENLIILANNSLSSNG | |
| | NVTESGCKECEELEEKNIKEFLQSFVHIVQMFINTS | |
| ErbB | MEFGLSWVFLVALFRGVQCEGLACHQLCARGHCWGPGPTQC | 90 |
| epitope- | VNCSQFLRGQECVEECRVLQGLPREYVNARHCLPCHPECQPQ | |
| P2A-IL15 | NGSVTCFGPEADQCVACAHYKDPPFCVARCPSGVKPDLSYM | |
| | PIWKFPDEEGACQPCPINCTHSCVDLDDKGCPAEQRASPLTSII | |
| | SAVVGILLVVVLGVVFGILIGGGGSGGATNFSLLKQAGDVEE | |
| | NPGPMRISKPHLRSISIQCYLCLLLNSHFLTEAGIHVFILGCFSA | |
| | GLPKTEANWVNVISDLKKIEDLIQSMHIDATLYTESDVHPSCK | |
| | VTAMKCFLLELQVISLESGDASIHDTVENLIILANNSLSSNGNV | |
| | TESGCKECEELEEKNIKEFLQSFVHIVQMFINTS | |
| HLA-E | HSLKYFHTSVSRPGRGEPRFISVGYVDDTQFVRFDNDAASPR | 91 |
| | MVPRAPWMEQEGSEYWDRETRSARDTAQIFRVNLRTLRGYY | |
| | NQSEAGSHTLQWMHGCELGPDGRFLRGYEQFAYDGKDYLTL | |
| | NEDLRSWTAVDTAAQISEQKSNDASEAEHQRAYLEDTCVEW | |
| | LHKYLEKGKETLLHLEPPKTHVTHHPISDHEATLRCWALGFY | |
| | PAEITLTWQQDGEGHTQDTELVETRPAGDGTFQKWAAVVVP | |
| | SGEEQRYTCHVQHEGLPEPVTLRWKPASQPTIPIVGIIAGLVLL | |
| | GSVVSGAVVAAVIWRKKSSGGKGGSYSKAEWSDSAQGSESH | |
| | SL | |
| HLA-G | MVVMAPRTLFLLLSGALTLTETWAVMAPRTLIL | 92 |
| Signal | | |
| Peptide | | |
| HLA-G | MVVMAPRTLFLLLSGALTLTETWAVMAPRTLILGGGGSGGG | 93 |
| Signal | GSGGGGSGGGGSIQRTPKIQVYSRHPAENGKSNFLNCYVSGF | |
| Peptide- | HPSDIEVDLLKNGERIEKVEHSDLSFSKDWSFYLLYYTEFTPTE | |
| B2M- | KDEYACRVNHVTLSQPKIVKWDRDMGGGGSGGGGSGGGGS | |
| HLA-E | GSHSLKYFHTSVSRPGRGEPRFISVGYVDDTQFVRFDNDAASP | |
| | RMVPRAPWMEQEGSEYWDRETRSARDTAQIFRVNLRTLRGY | |
| | YNQSEAGSHTLQWMHGCELGPDGRFLRGYEQFAYDGKDYL | |
| | TLNEDLRSWTAVDTAAQISEQKSNDASEAEHQRAYLEDTCVE | |
| | WLHKYLEKGKETLLHLEPPKTHVTHHPISDHEATLRCWALGF | |
| | YPAEITLTWQQDGEGHTQDTELVETRPAGDGTFQKWAAVVV | |
| | PSGEEQRYTCHVQHEGLPEPVTLRWKPASQPTIPIVGIIAGLVL | |
| | LGSVVSGAVVAAVIWRKKSSGGKGGSYSKAEWSDSAQGSES | |
| | HSL | |
| HLA-G | ATGGTGGTCATGGCCCCTAGAACACTGTTCCTGCTGCTGTC | 94 |
| Signal | TGGCGCCCTGACACTGACAGAGACATGGGCCGTGATGGCC | |
| Peptide- | CCCAGAACCCTGATCCTGGGCGGCGGTGGTTCAGGCGGAG | |
| B2M- | GAGGTTCAGGAGGAGGGGGTAGTGGAGGTGGTGGTTCTAT | |
| HLA-E | CCAGCGGACCCCTAAGATCCAGGTGTACAGCAGACACCCC | |
| | GCCGAGAACGGCAAGAGCAACTTCCTGAACTGCTACGTGT | |
| | CCGGCTTTCACCCCAGCGACATTGAGGTGGACCTGCTGAA | |
| | GAACGGCGAGCGGATCGAGAAGGTGGAACACAGCGATCT | |
| | GAGCTTCAGCAAGGACTGGTCCTTCTACCTGCTGTACTACA | |
| | CCGAGTTCACCCCTACCGAGAAGGACGAGTACGCCTGCAG | |
| | AGTGAACCACGTGACACTGAGCCAGCCTAAGATCGTGAAG | |
| | TGGGATCGCGATATGGGCGGAGGCGGATCTGGTGGCGGAG | |
| | GAAGTGGCGGCGGAGGATCTGGCTCCCACTCCTTGAAGTA | |
| | TTTCCACACTTCCGTGTCCCGGCCCGGCCGCGGGGAGCCCC | |
| | GCTTCATCTCTGTGGGCTACGTGGACGACACCCAGTTCGTG | |
| | CGCTTCGACAACGACGCCGCGAGTCCGAGGATGGTGCCGC | |
| | GGGCGCCGTGGATGGAGCAGGAGGGGTCAGAGTATTGGGA | |
| | CCGGGAGACACGGAGCGCCAGGGACACCGCACAGATTTTC | |
| | CGAGTGAATCTGCGGACGCTGCGCGGCTACTACAATCAGA | |
| | GCGAGGCCGGGTCTCACACCCTGCAGTGGATGCATGGCTG | |
| | CGAGCTGGGGCCCGACGGGCGCTTCCTCCGCGGGTATGAA | |
| | CAGTTCGCCTACGACGGCAAGGATTATCTCACCCTGAATGA | |
| | GGACCTGCGCTCCTGGACCGCGGTGGACACGGCGGCTCAG | |
| | ATCTCCGAGCAAAAGTCAAATGATGCCTCTGAGGCGGAGC | |
| | ACCAGAGAGCCTACCTGGAAGACACATGCGTGGAGTGGCT | |
| | CCACAAATACCTGGAGAAGGGGAAGGAGACGCTGCTTCAC | |
| | CTGGAGCCCCCAAAGACACACGTGACTCACCACCCCATCT | |
| | CTGACCATGAGGCCACCCTGAGGTGCTGGGCCCTGGGCTTC | |
| | TACCCTGCGGAGATCACACTGACCTGGCAGCAGGATGGGG | |
| | AGGGCCATACCCAGGACACGGAGCTCGTGGAGACCAGGCC | |
| | TGCAGGGGATGGAACCTTCCAGAAGTGGGCAGCTGTGGTG | |
| | GTGCCTTCTGGAGAGGAGCAGAGATACACGTGCCATGTGC | |
| | AGCATGAGGGGCTACCCGAGCCCGTCACCCTGAGATGGAA | |
| | GCCGGCTTCCCAGCCCACCATCCCCATCGTGGGCATCATTG | |
| | CTGGCCTGGTTCTCCTTGGATCTGTGGTCTCTGGAGCTGTG | |
| | GTTGCTGCTGTGATATGGAGGAAGAAGAGCTCAGGTGGAA | |
| | AAGGAGGGAGCTACTCTAAGGCTGAGTGGAGCGACAGTGC | |
| | CCAGGGGTCTGAGTCTCACAGCTTGTAA | |
| HLA-G | HSMRYFSAAVSRPGRGEPRFIAMGYVDDTQFVRFDSDSACPR | 95 |
| | MEPRAPWVEQEGPEYWEEETRNTKAHAQTDRMNLQTLRGY | |
| | YNQSEASSHTLQWMIGCDLGSDGRLLRGYEQYAYDGKDYLA | |
| | LNEDLRSWTAADTAAQISKRKCEAANVAEQRRAYLEGTCVE | |
| | WLHRYLENGKEMLQRADPPKTHVTHHPVFDYEATLRCWAL | |
| | GFYPAEIILTWQRDGEDQTQDVELVETRPAGDGTFQKWAAV | |
| | VVPSGEEQRYTCHVQHEGLPEPLMLRWKQSSLPTIPIMGIVAG | |
| | LVVLAAVVTGAAVAAVLWRKKSSD | |
| HLA-G | MVVMAPRTLFLLLSGALTLTETWARIIPRHLQLGGGGSGGGG | 96 |
| Signal | SIQRTPKIQVYSRHPAENGKSNFLNCYVSGFHPSDIEVDLLKN | |
| Peptide- | GERIEKVEHSDLSFSKDWSFYLLYYTEFTPTEKDEYACRVNH | |
| B2M- | VTLSQPKIVKWDRDMGGGGSGGGGSGGGGSGSHSMRYFSAA | |
| HLA-G | VSRPGRGEPRFIAMGYVDDTQFVRFDSDSACPRMEPRAPWVE | |
| | QEGPEYWEEETRNTKAHAQTDRMNLQTLRGYYNQSEASSHT | |
| | LQWMIGCDLGSDGRLLRGYEQYAYDGKDYLALNEDLRSWT | |
| | AADTAAQISKRKCEAANVAEQRRAYLEGTCVEWLHRYLENG | |
| | KEMLQRADPPKTHVTHHPVFDYEATLRCWALGFYPAEIILTW | |
| | QRDGEDQTQDVELVETRPAGDGTFQKWAAVVVPSGEEQRYT | |
| | CHVQHEGLPEPLMLRWKQSSLPTIPIMGIVAGLVVLAAVVTG | |
| | AAVAAVLWRKKSSD | |
| HLA-G | GCCACCATGGTGGTCATGGCGCCCCGAACCCTCTTCCTGCT | 97 |
| Signal | GCTCTCGGGGGCCCTGACCCTGACCGAGACCTGGGCGCGG | |
| Peptide- | ATCATTCCCCGACATCTGCAACTGGGAGGCGGCGGTTCAG | |
| B2M- | GAGGGGGCGGATCGATCCAACGCACCCCCAAGATCCAGGT | |
| HLA-G | CTACTCCAGACACCCGGCCGAAAACGGAAAGTCGAACTTC | |
| | CTGAACTGCTATGTGTCAGGATTCCACCCGTCCGACATCGA | |
| | GGTGGACCTCCTGAAGAACGGCGAACGCATTGAGAAGGTC | |
| | GAGCACTCCGATCTGTCGTTCTCCAAGGACTGGTCCTTCTA | |
| | CCTTCTCTACTATACCGAATTCACCCCGACCGAGAAGGACG | |
| | AATACGCCTGCCGGGTCAACCACGTGACCCTGAGCCAGCC | |
| | AAAGATCGTGAAATGGGACCGCGATATGGGAGGAGGAGG | |
| | TTCCGGCGGAGGAGGAAGCGGAGGCGGAGGTTCCGGCTCC | |
| | CACTCCATGAGGTATTTCAGCGCCGCCGTGTCCCGGCCTGG | |
| | CCGCGGAGAGCCTCGCTTCATCGCCATGGGATACGTGGAC | |
| | GACACCCAGTTCGTCAGATTCGACAGCGACAGCGCCTGTC | |
| | CTCGGATGGAACCTAGAGCACCTTGGGTCGAGCAAGAGGG | |
| | CCCTGAGTACTGGGAAGAAGAGACACGGAACACCAAGGCT | |
| | CACGCCCAGACCGACAGAATGAACCTGCAGACCCTGCGGG | |
| | GCTACTACAATCAGTCTGAGGCCAGCAGCCATACTCTGCA | |
| | GTGGATGATCGGCTGCGATCTGGGCTCTGATGGCAGACTG | |
| | CTGAGAGGCTACGAGCAGTACGCCTACGACGGCAAGGATT | |
| | ATCTGGCCCTGAACGAGGACCTGCGGTCTTGGACAGCTGC | |
| | CGATACAGCCGCTCAGATCAGCAAGAGAAAGTGCGAGGCC | |
| | GCCAATGTGGCCGAACAGAGAAGGGCTTACCTGGAAGGCA | |
| | CCTGTGTGGAATGGCTGCACAGATACCTGGAAAACGGCAA | |
| | AGAGATGCTGCAGCGGGCCGATCCTCCTAAGACACATGTG | |
| | ACCCACCATCCTGTGTTCGACTACGAGGCCACACTGAGATG | |
| | TTGGGCCCTGGGCTTTTACCCTGCCGAGATCATCCTGACCT | |
| | GGCAGCGAGATGGCGAGGATCAGACCCAGGATGTGGAACT | |
| | GGTGGAAACCAGACCTGCCGGCGACGGCACCTTTCAGAAA | |
| | TGGGCTGCTGTGGTGGTGCCCAGCGGAGAGGAACAGAGAT | |
| | ACACCTGTCACGTGCAGCACGAGGGACTGCCTGAACCTCT | |
| | GATGCTGAGATGGAAGCAGAGCAGCCTGCCTACAATCCCC | |
| | ATCATGGGAATCGTGGCCGGACTGGTGGTTCTGGCCGCTGT | |
| | TGTTACAGGTGCTGCAGTGGCTGCCGTGCTGTGGCGGAAG | |
| | AAAAGCAGCGACTGA | |
| CAG | ATTGATTATTGACTAGTTATTAATAGTAATCAATTACGGGG | 98 |
| Promoter | TCATTAGTTCATAGCCCATATATGGAGTTCCGCGTTACATA | |
| | ACTTACGGTAAATGGCCCGCCTGGCTGACCGCCCAACGAC | |
| | CCCCGCCCATTGACGTCAATAATGACGTATGTTCCCATAGT | |
| | AACGCCAATAGGGACTTTCCATTGACGTCAATGGGTGGAC | |
| | TATTTACGGTAAACTGCCCACTTGGCAGTACATCAAGTGTA | |
| | TCATATGCCAAGTACGCCCCCTATTGACGTCAATGACGGTA | |
| | AATGGCCCGCCTGGCATTATGCCCAGTACATGACCTTATGG | |
| | GACTTTCCTACTTGGCAGTACATCTACGTATTAGTCATCGC | |
| | TATTACCATGGGTCGAGGTGAGCCCCACGTTCTGCTTCACT | |
| | CTCCCCATCTCCCCCCCCTCCCCACCCCCAATTTTGTATTTA | |
| | TTTATTTTTTAATTATTTTGTGCAGCGATGGGGGCGGGGGG | |
| | GGGGGGGGCGCGCGCCAGGCGGGGCGGGGCGGGGCGAGG | |
| | GGCGGGGCGGGGCGAGGCGGAGAGGTGCGGCGGCAGCCA | |
| | ATCAGAGCGGCGCGCTCCGAAAGTTTCCTTTTATGGCGAGG | |
| | CGGCGGCGGCGGCGGCCCTATAAAAAGCGAAGCGCGCGGC | |
| | GGGCGGGAGTCGCTGCGTTGCCTTCGCCCCGTGCCCCGCTC | |
| | CGCGCCGCCTCGCGCCGCCCGCCCCGGCTCTGACTGACCGC | |
| | GTTACTCCCACAGGTGAGCGGGCGGGACGGCCCTTCTCCTC | |
| | CGGGCTGTAATTAGCGCTTGGTTTAATGACGGCTCGTTTCT | |
| | TTTCTGTGGCTGCGTGAAAGCCTTAAAGGGCTCCGGGAGG | |
| | GCCCTTTGTGCGGGGGGGAGCGGCTCGGGGGGTGCGTGCG | |
| | TGTGTGTGTGCGTGGGGAGCGCCGCGTGCGGCCCGCGCTG | |
| | CCCGGCGGCTGTGAGCGCTGCGGGCGCGGCGCGGGGCTTT | |
| | GTGCGCTCCGCGTGTGCGCGAGGGGAGCGCGGCCGGGGGC | |
| | GGTGCCCCGCGGTGCGGGGGGGCTGCGAGGGGAACAAAG | |
| | GCTGCGTGCGGGGTGTGTGCGTGGGGGGGTGAGCAGGGGG | |
| | TGTGGGCGCGGCGGTCGGGCTGTAACCCCCCCCTGCACCCC | |
| | CCTCCCCGAGTTGCTGAGCACGGCCCGGCTTCGGGTGCGG | |
| | GGCTCCGTGCGGGGCGTGGCGCGGGGCTCGCCGTGCCGGG | |
| | CGGGGGGTGGCGGCAGGTGGGGGTGCCGGGCGGGGCGGG | |
| | GCCGCCTCGGGCCGGGGAGGGCTCGGGGGAGGGGCGCGG | |
| | CGGCCCCGGAGCGCCGGCGGCTGTCGAGGCGCGGCGAGCC | |
| | GCAGCCATTGCCTTTTATGGTAATCGTGCGAGAGGGCGCA | |
| | GGGACTTCCTTTGTCCCAAATCTGGCGGAGCCGAAATCTGG | |
| | GAGGCGCCGCCGCACCCCCTCTAGCGGGCGCGGGCGAAGC | |
| | GGTGCGGCGCCGGCAGGAAGGAAATGGGCGGGGAGGGCC | |
| | TTCGTGCGTCGCCGCGCCGCCGTCCCCTTCTCCATCTCCAG | |
| | CCTCGGGGCTGCCGCAGGGGGACGGCTGCCTTCGGGGGGG | |
| | ACGGGGCAGGGCGGGGTTCGGCTTCTGGCGTGTGACCGGC | |
| | GGGATATCTACGAAGCGGCCGCCCTCTGCTAACCATGTTCA | |
| | TGCCTTCTTCTTTTTCCTACAGCTCCTGGGCAACGTGCTGGT | |
| | TATTGTGCTGTCTCATCATTTTGGCAAA | |
| SV40 | AACTTGTTTATTGCAGCTTATAATGGTTACAAATAAAGCAA | 99 |
| Termin- | TAGCATCACAAATTTCACAAATAAAGCATTTTTTTCACTGC | |
| ator | ATTCTAGTTGTGGTTTGTCCAAACTCATCAATGTATCTT | |
| tEGFR- | ATGAGGCCCTCAGGCACTGCCGGGGCCGCCCTCCTGGCCCT | 100 |
| P2A-IL15 | GTTAGCCGCTTTGTGTCCAGCAAGCCGCGCCGGAGTGCGG | |
| | AAATGTAAGAAATGCGAAGGACCCTGCCGGAAGGTATGCA | |
| | ACGGCATTGGGATTGGCGAATTCAAGGACAGCCTGAGCAT | |
| | TAATGCTACAAACATCAAGCACTTTAAGAATTGCACCAGC | |
| | ATTAGCGGCGATCTGCATATACTGCCAGTGGCTTTCCGAGG | |
| | CGACTCTTTTACTCATACCCCTCCGCTGGACCCTCAAGAGC | |
| | TGGACATTCTCAAGACTGTGAAGGAAATTACGGGGTTTCTG | |
| | CTCATTCAGGCCTGGCCTGAAAACCGCACGGATTTGCATGC | |
| | CTTTGAGAATCTGGAAATAATCAGAGGCCGGACGAAACAG | |
| | CATGGCCAGTTCAGCCTCGCGGTCGTCTCTTTGAATATTAC | |
| | GTCACTCGGCCTCAGGTCCCTCAAAGAGATTTCTGATGGCG | |
| | ATGTCATCATCTCTGGTAATAAGAATCTGTGTTACGCAAAT | |
| | ACCATCAATTGGAAGAAGCTCTTTGGGACCTCAGGTCAAA | |
| | AGACTAAAATTATCTCCAACCGCGGCGAGAACAGCTGTAA | |
| | GGCTACAGGCCAGGTTTGCCACGCGCTCTGCTCCCCAGAG | |
| | GGTTGCTGGGGGCCTGAGCCAAGGGATTGCGTTTCATGTCG | |
| | CAACGTGTCTCGGGGCAGAGAATGCGTGGATAAATGTAAC | |
| | CTCTTAGAGGGCGAACCTCGCGAGTTTGTTGAGAACTCAG | |
| | AATGTATACAGTGCCACCCCGAATGTCTTCCTCAGGCCATG | |
| | AATATCACATGCACCGGACGCGGACCAGACAACTGTATCC | |
| | AATGTGCTCACTACATTGACGGACCTCATTGTGTGAAAACA | |
| | TGCCCCGCAGGAGTTATGGGAGAAAACAACACCCTCGTTT | |
| | GGAAATATGCCGATGCAGGTCACGTATGTCACCTGTGCCA | |
| | CCCAAACTGCACTTATGGGTGCACCGGGCCGGGCCTGGAG | |
| | GGGTGCCCTACGAATGGACCAAAAATTCCCAGTATTGCAA | |
| | CTGGGATGGTCGGGGCACTGTTGTTGCTGCTTGTGGTTGCC | |
| | CTCGGGATAGGCCTGTTTATGTCTGGCTCCGGCGCCACCAA | |
| | TTTCAGCCTGCTGAAACAGGCAGGCGACGTCGAAGAAAAT | |
| | CCAGGACCAATGCGAATATCAAAACCACACTTGCGCAGCA | |
| | TTTCTATACAGTGCTATTTGTGCTTGTTGCTGAACTCTCACT | |
| | TCCTCACAGAGGCTGGGATACACGTTTTCATACTTGGATGT | |
| | TTTTCAGCTGGGCTGCCGAAGACAGAGGCGAATTGGGTGA | |
| | ATGTAATTTCAGACCTCAAGAAGATCGAGGATCTCATCCA | |
| | GTCCATGCACATCGACGCTACTCTGTACACAGAGAGCGAT | |
| | GTCCACCCTTCTTGTAAGGTTACCGCCATGAAATGCTTCCT | |
| | TTTGGAACTCCAAGTCATCTCATTGGAATCAGGGGATGCGT | |
| | CCATTCATGACACCGTGGAAAACCTGATAATACTGGCTAA | |
| | CAACAGCTTGTCAAGTAATGGGAATGTTACTGAGTCCGGTT | |
| | GTAAAGAATGTGAAGAGCTGGAGGAGAAGAACATTAAGG | |
| | AATTTTTGCAATCTTTTGTACATATTGTTCAGATGTTTATTA | |
| | ACACAAGC | |
| (G4S)2 | GGGGSGGGGS | 101 |
| Linker | | |
| (G4S)6 | GGGGSGGGGSGGGGSGGGGSGGGGSGGGGS | 102 |
| Linker | | |
| (G4S)7 | GGGGSGGGGSGGGGSGGGGSGGGGSGGGGSGGGGS | 103 |
| Linker | | |
| (G4S)8 | GGGGSGGGGSGGGGSGGGGSGGGGSGGGGSGGGGSGGGGS | 104 |
| Linker | | |

D. HLA Expression

In certain embodiments, the iPSC of the application can be further modified by introducing an exogenous polynucleotide encoding one or more proteins related to immune evasion, such as non-classical HLA class I proteins (e.g., HLA-E and HLA-G). In particular, disruption of the B2M gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol. 2017; 35(8): 765-772).

In certain embodiments, the iPSC or derivative cell thereof comprises a polypeptide encoding at least one of a human leukocyte antigen E (HLA-E) and human leukocyte antigen G (HLA-G). In a particular embodiment, the HLA-E comprises an amino acid sequence at least 90%, such as at least 90%, 91%, 92%, 93%, 94%, 95%, 96%, 97%, 98%, 99% or 100%, identical to SEQ ID NO: 91, preferably the amino acid sequence of SEQ ID NO: 91. In a particular embodiment, the HLA-G comprises an amino acid sequence at least 90%, such as at least 90%, 91%, 92%, 93%, 94%, 95%, 96%, 97%, 98%, 99% or 100%, identical to SEQ ID NO: 95, preferably SEQ ID NO: 95.

In certain embodiments, the exogenous polynucleotide encodes a polypeptide comprising a signal peptide operably linked to a mature B2M protein that is fused to an HLA-E via a linker. In a particular embodiment, the exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 93.

In other embodiments, the exogenous polynucleotide encodes a polypeptide comprising a signal peptide operably linked to a mature B2M protein that is fused to an HLA-G via a linker. In a particular embodiment, the exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 96.
E. Other Optional Genome Edits

In other embodiments of the above described cell, the genomic editing employing the RNP complex of this disclosure may comprise insertions of one or more exogenous polynucleotides encoding other additional artificial cell death polypeptides proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof. Other transgene inserts may include those encoding PET reporters, homeostatic cytokines, and inhibitory checkpoint inhibitory proteins such as PD1, PD-L1, and CTLA4 as well as proteins that target the CD47/signal regulatory protein alpha (SIRPa) axis.

### V. Regulatory Elements

In certain embodiments, the polynucleotide encoding the MAD7 nuclease, the gRNA, or the exogenous polynucleotide for insertion is operably linked to at least a regulatory element. The regulatory element can be capable of mediating expression of the MAD7, gRNA, and/or the transgene in the host cell. Regulatory elements include, but are not limited to, promoters, enhancers, initiation sites, polyadenylation (polyA) tails, IRES elements, response elements, and termination signals.

In some embodiments, the exogenous polynucleotides for insertion are operatively linked to (1) one or more exogenous promoters comprising CMV, EFla, PGK, CAG, UBC, SV40, human beta actin, or other constitutive, inducible, temporal-, tissue-, or cell type-specific promoters; or (2) one or more endogenous promoters comprised in the selected sites such as AAVS1, B2M, CIITA, NKG2A, TRAC, CD70, CD38, CD33, or CLYBL, or other locus meeting the criteria of a genome safe harbor.

In some embodiments, the promoter is a CAG promoter. In some embodiments, the CAG promoter comprises the polynucleotide sequence at least 90%, such as at least 90%, 91%, 92%, 93%, 94%, 95%, 96%, 97%, 98% or 100%, identical to SEQ ID NO: 98.

In some embodiment, the exogenous polynucleotides for insertion are placed operably under the control of a Kozak consensus sequence. In some embodiments, the Kozak sequence comprises the polynucleotide sequence of GCCACC, or a variant thereof.

In certain embodiments, the exogenous polynucleotides for insertion are operatively linked to a terminator/polyadenylation signal. In some embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 99. Other terminator sequences can also be used, examples of which include, but are not limited to BGH, hGH, and PGK.

### VI. Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the application.

### VII. Methods of Use

In another general aspect, the application provides a method of treating a disease or a condition in a subject in need thereof. The methods comprise administering to the subject in need thereof a therapeutically effective amount of cells of the application and/or a composition of the application. In certain embodiments, the disease or condition is cancer. The cancer can, for example, be a solid or a liquid cancer. The cancer, can, for example, be selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. In a preferred embodiment, the cancer is a non-Hodgkin's lymphoma (NHL).

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered transarterially, subcutaneously, intradermaly, intratumorally, intranodally, intramedullary, intramuscularly, inrapleurally, by intravenous (i.v.) injection, or intraperitoneally. In certain embodiments, the cells of the application can be administered with or without lymphodepletion of the subject.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

### EXAMPLES

The following examples are provided to further describe some of the embodiments disclosed herein. The examples are intended to illustrate, not to limit, the disclosed embodiments.

### Example 1. Site-Specific Engineering of iPSCs Using a Two-Step Transfection Process

Day 1: Lipofectamine-stem transfection of donor pDNA into iPSCs

100 μM stock H1152 Rho inhibitor solution is added to the T-75 flask containing iPSCs at approximately 70% confluency to a concentration of 1 μM. Cells are incubated at 37° C., 5% CO₂, low O₂ incubator for at least 1 hour. During the incubation, vitronectin coated T75 flasks are allowed to come to room temperature for at least 15 minutes. The coating solution is aspirated from each flask and replace with 10 mL Complete Essential 8 Media+1 μM H1152. The plate is placed in a 37° C., 5% CO₂, low O₂ incubator until use. After the incubation, the media is aspirated from the T-75 flask containing iPSCs, 7 mL of 1× DPBS is added along the side of the flask and gently swirled to wash. DPBS is aspirated and 2 mL of TrypLE Select is added directly to the cells. The cells are incubated at 37° C. for 3 to 5 minutes followed by the addition of 10 mL of Complete Essential 8 media to the flask. Cells are lifted off the plate by pipetting and then transferred into a sterile 50 mL conical tube. Cells are centrifuged at 200×g for 5 minutes. The supernatant is aspirated and cells re-suspended in 10 mL of Complete Essential 8 Medium. Cells are counted using the NC-200 NucleoCounter. To the T-75 flask, 2E6 cells are seeded in each flask. Cells are incubated at 37° C., 5% CO₂, low 02 incubator until needed for transfection. Transfection mixes are set up as listed below in sterile 15 mL centrifuge tube according to the table below, scaling up as necessary:

Tube #1

| | | |
|---|---|---|
| Opti-MEM | 1250 | μl |
| Lipofectamine Stem | 50 | μl |

Tube #2

| | | |
|---|---|---|
| Opti-MEM | 1250 | μl |
| pDNA | 5 | μg |

Tube 1 and tube 2 are mixed by adding components of tube 2 into tube 1 and then incubated at ambient temperature for 10 minutes. The entire mix is added dropwise into appropriate flasks. The flasks are gently rocked and placed in a 37° C., 5% C02, low 02 incubator.

Day 2: Feeding iPSCs

Complete Essential 8 Medium is brought to ambient temperature (≥15 minutes). Spent medium from iPSC cultures is replace with 14 mL fresh Complete Essential 8 Medium per vessel and cultures are returned to 37° C. hypoxic 5% CO₂ humidified incubator immediately after feeding is complete. Feed/media exchange on iPSC cultures the day of passaging is not performed as this will significantly decrease detachment of colonies.

Day 3: Generation of Ribonucleoprotein (RNP) Complex

Electroporation is performed 40-48 hours post-transfection of iPSCs with donor pDNA. The following is combined in a sterile PCR tube and mixed well (multiply volumes for the appropriate number of conditions+1 for overage)
1.4 μL 1× DPBS
1.6 μL 100 μM Alt-R CRISPR-MAD7 crRNA
2 μL Alt-R MAD7 Ultra Nuclease

The solution is centrifuged briefly and incubated at ambient temperature for 10-20 mins and then stored at 2-8° C. until needed for electroporation.

The spent media is aspirated from the T-75 flask containing cells and 7 mL of 1× DPBS is added to wash. 1× DPBS is aspirated and replaced with 2 mL of TrypLE. The flask is placed in low 02 incubator at 37° C., 5% CO₂ for 3-5 mins followed by the addition of 10 mL of Complete E8 media and pipetted up and down 3-4 times to dislodge cells. Cells are transferred to a 50 mL conical and centrifuged at 200×g for 5 minutes. During the centrifugation, the appropriate number of coated 6 well plates are prepared by aspirating the coating solution from each well and addition of 2 mL Complete Essential 8 Media+1 μM H1152 to each well. The supernatant is aspirated and the cells are re-suspended in 10 mL of cold Opti-MEM media followed by another centrifugation at 200×g for 5 minutes. The supernatant is aspirated and cells resuspended again in 10 mL cold Opti-MEM media. The cells are counted on the NC-200 Cell Counter and recorded.

The cells are centrifuged at 200×g for 5 minutes and resuspended in Opti-MEM previously equilibrated to ambient temperature at a concentration of 2×10⁶ cells per mL. BTX ECM-830 Electroporator is set to:
150V
10 ms
1 pulse

For each electroporation add the following into a BTX electroporation cuvette with a 2 mm gap width.
5 μl RNP complex
1.4 μL Cpf1 electroporation enhancer
200 μl of cells

The cuvette is tapped to ensure that all the contents fall to the bottom and placed in the electroporation safety stand, the dome closed, and start button pushed.

A sterile transfer pipette provided with each cuvette is used to add the cells dropwise to the appropriate well of the prepared 6-well plate and then placed in low O₂ incubator at 37° C., 5% CO₂.

### Example 2. Editing of AAVS1 Locus

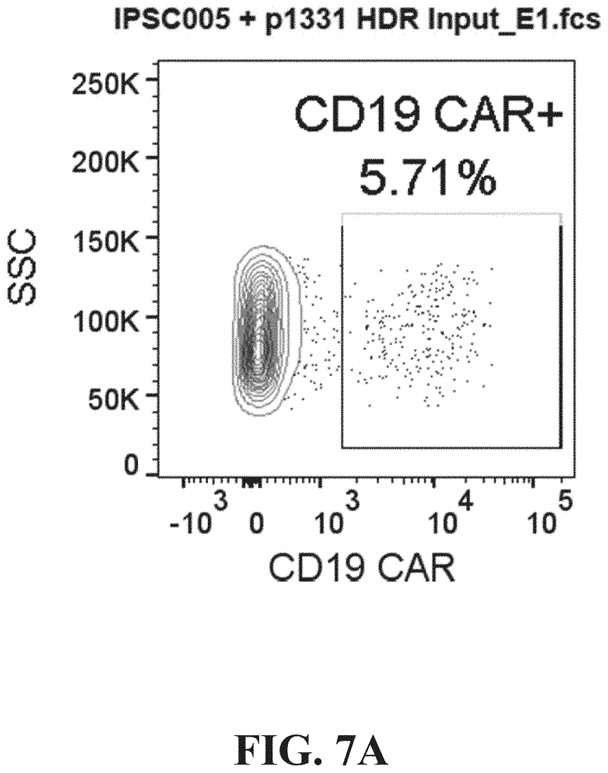

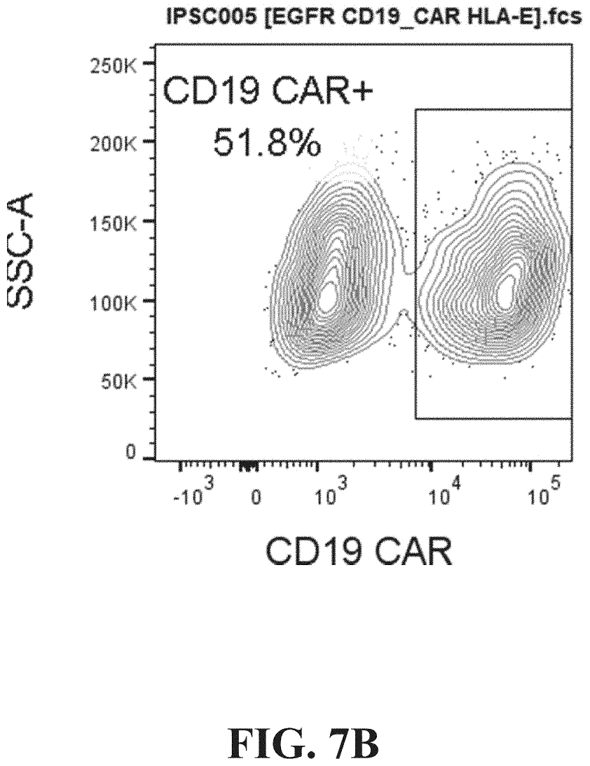

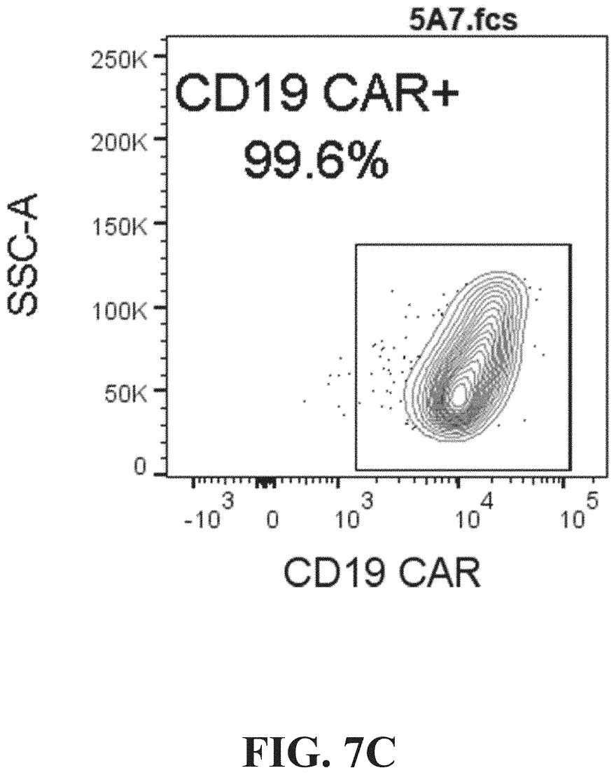

CAR transgene donor plasmid was specifically engineered to insert a CAR into the AAVS1 site. FIG. 7A depicts flow cytometry analysis of bulk population of cells post-engineering. FIG. 7B depicts flow cytometry analysis of cells post-sorting for CAR positive cells. FIG. 7C depicts flow cytometry analysis of CAR positive single cell clones.

### Example 3. Editing of B2M Locus

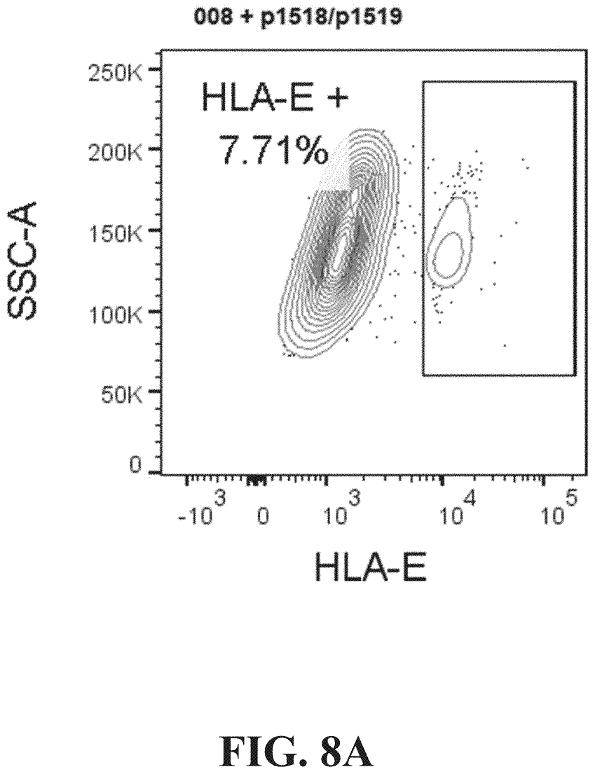

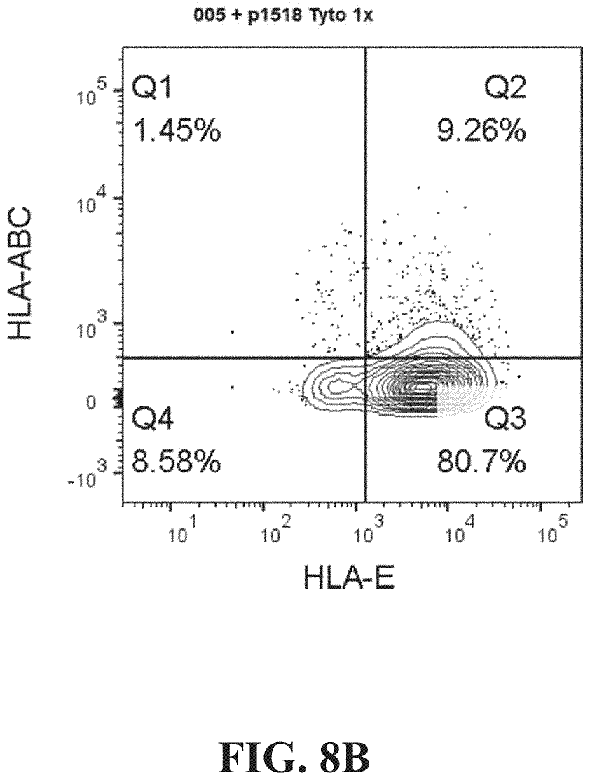

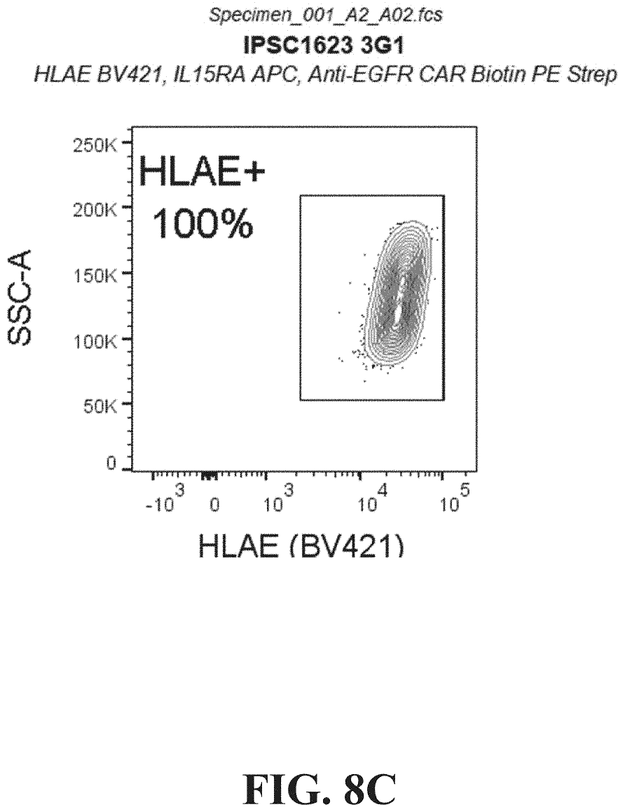

HLA-E transgene donor plasmid was specifically engineered to insert HLA-E into the B2M site. FIG. 8A depicts flow cytometry analysis of bulk population of cells post-engineering. FIG. 8B depicts flow cytometry analysis of cells post-sorting for HLA-E positive, B2M negative cells. FIG. 8C depicts flow cytometry analysis of HLA-E positive, B2M negative single cell clones.

### Example 4. Editing of CIITA Locus

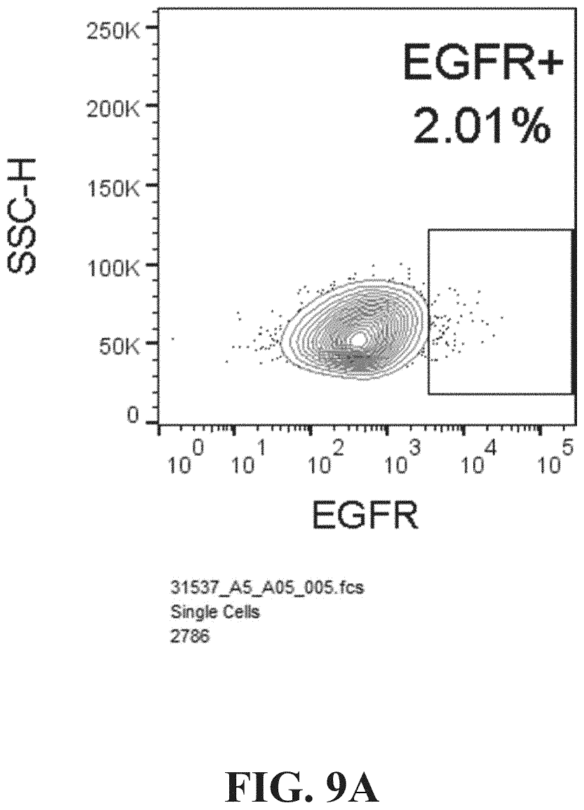

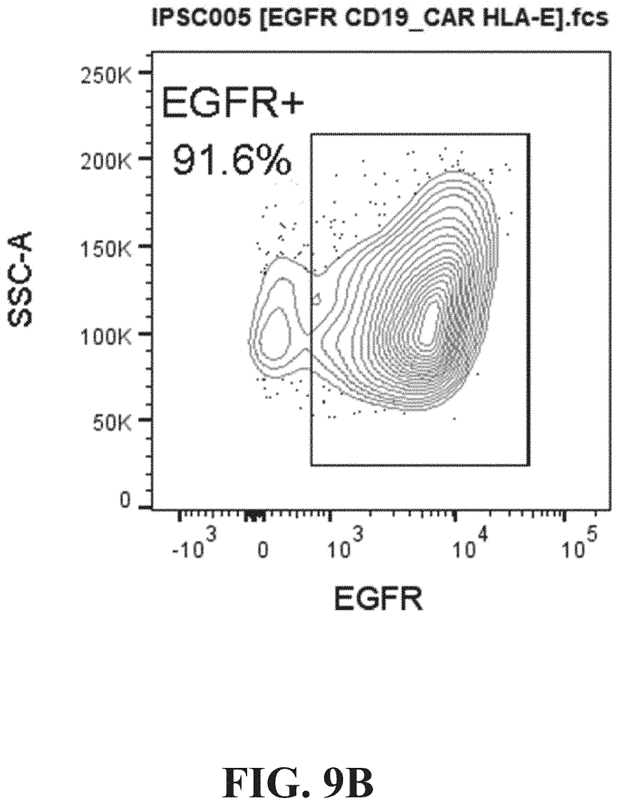

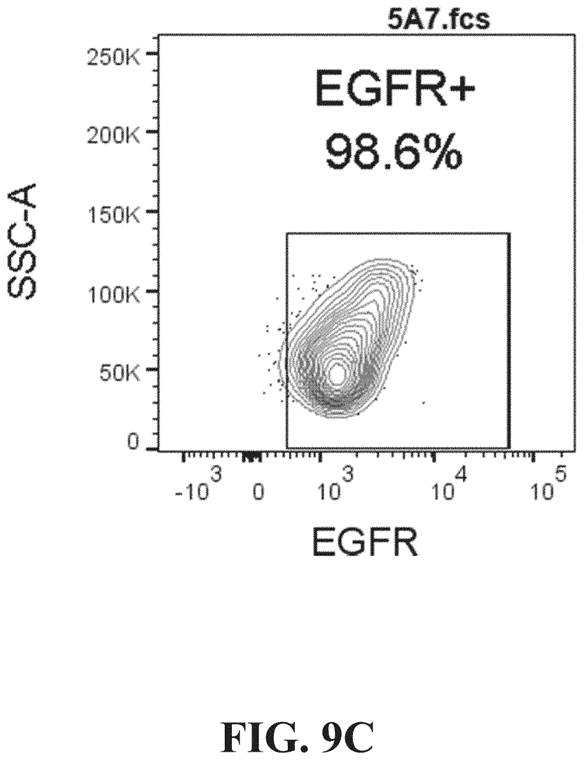

EGFR transgene donor plasmid was specifically engineered to insert EGFR into the CIITA site. FIG. 9A depicts flow cytometry analysis of bulk population of cells post-engineering. FIG. 9B depicts flow cytometry analysis of cells post-sorting for EGFR cells. FIG. 9C depicts flow cytometry analysis of EGFR positive single cell clones.

### Example 5. Editing of CYBYL Locus

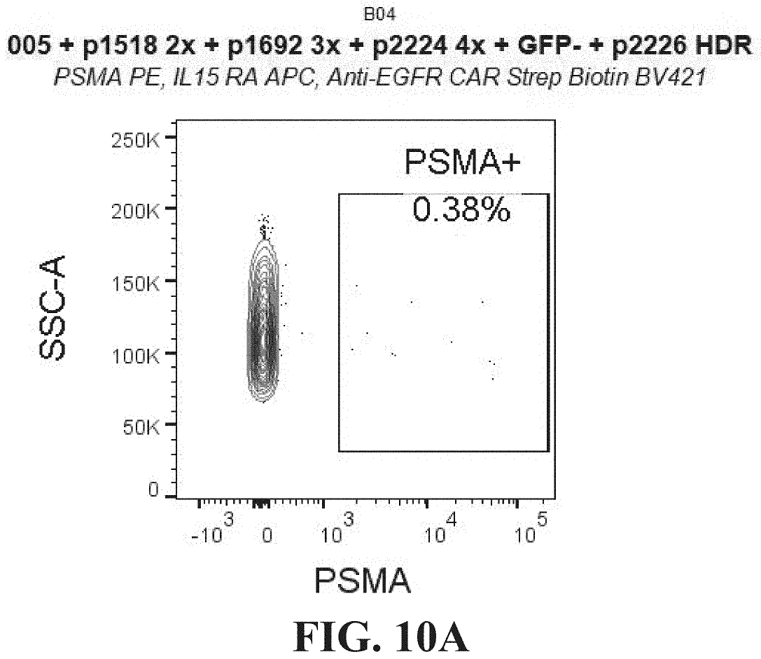

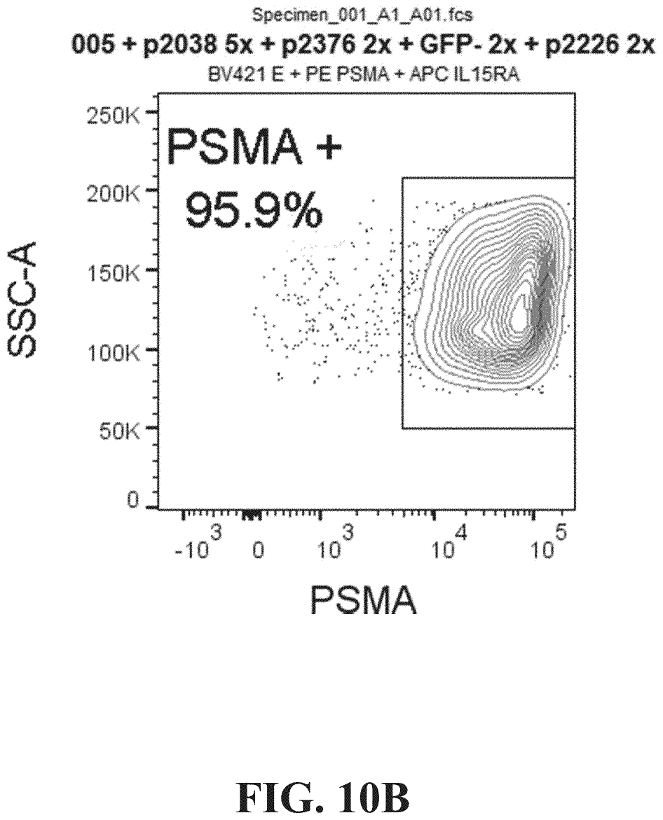

PSMA transgene donor plasmid was specifically engineered to insert PSMA into the CLYBL site. FIG. 10A depicts flow cytometry analysis of bulk population of cells post-engineering. FIG. 10B depicts flow cytometry analysis of cells post-sorting for PSMA positive cells.

### Example 6. Editing of NKG2A Locus

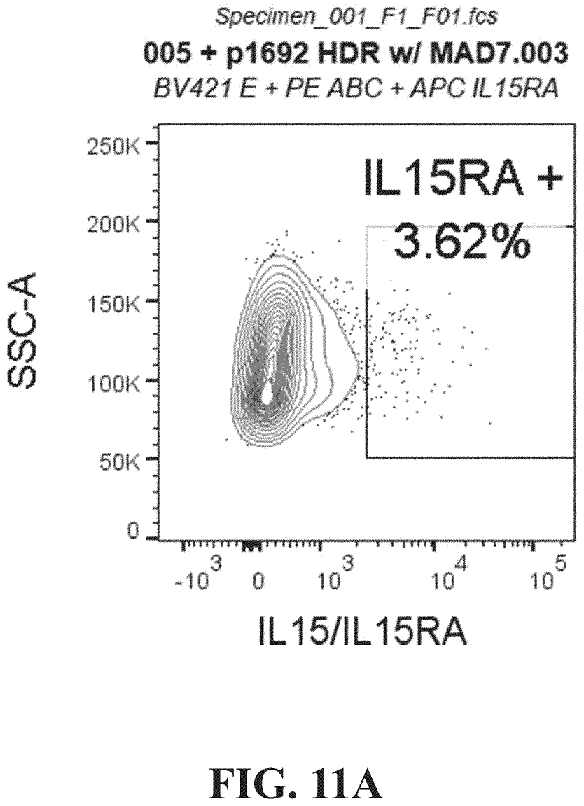

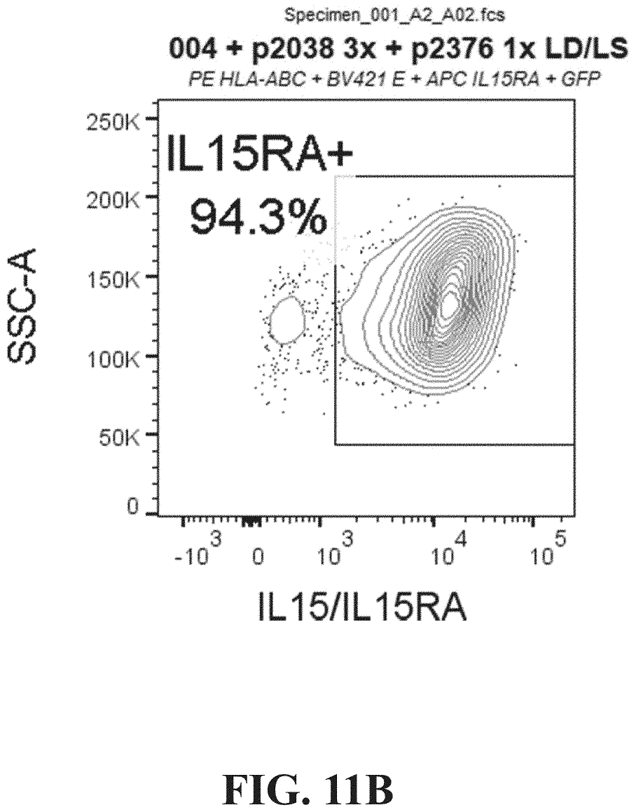

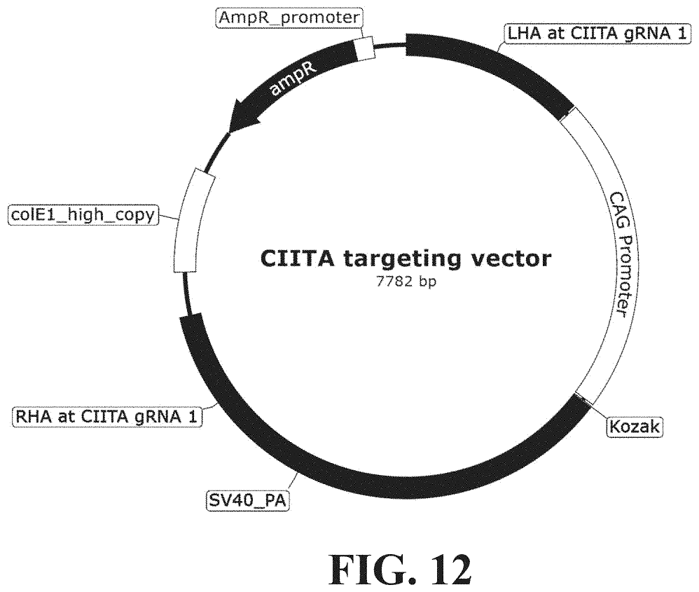

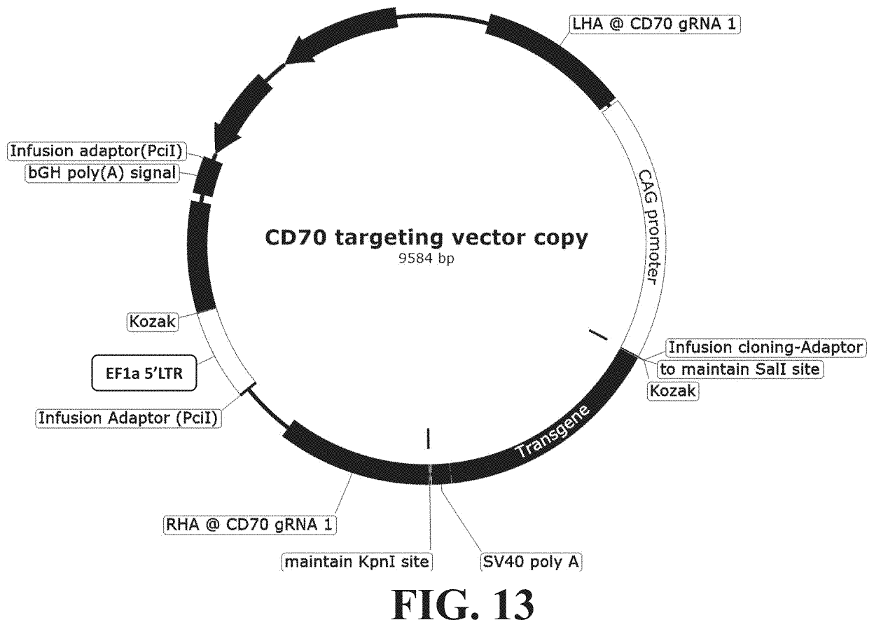

An IL15-IL15RA transgene donor plasmid was specifically engineered to insert IL15-IL15RA into the NKG2A site. FIG. 11A depicts flow cytometry analysis of bulk population of cells post-engineering. FIG. 11B depicts flow cytometry analysis of cells post-sorting for IL-15-IL15RA positive cells.

The present disclosure is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A MAD7/gRNA ribonucleoprotein (RNP) complex composition for insertion of a transgene, comprising:
(I) a MAD7 nuclease;
(II) a guide RNA (gRNA) specific for the MAD7 nuclease, wherein the gRNA comprises a guide sequence capable of hybridizing to a target sequence of an AAVS1, B2M, CIITA, NKG2A, TRAC, CD70, CD38, CD33, or CLYBL loci in a cell, wherein the guide sequence is selected from SEQ ID NOs: 120-130, wherein when the gRNA is complexed with the MAD7 nuclease, the guide sequence directs sequence-specific binding of the MAD7 nuclease to the target sequence; and
(III) a transgene vector comprising: (1) left and right polynucleotide sequences that are homologous to left and right arms of the target sequence of the AAVS1, B2M, CIITA, NKG2A, TRAC, CD70, CD38, CD33, or CLYBL loci, (2) a promoter which is operably linked to (3) a polynucleotide sequence encoding the transgene, and (4) a transcription terminator sequence.

2. The composition according to claim 1, wherein the transgene comprises a sequence encoding a chimeric antigen receptor (CAR), optionally wherein the CAR is specific for a tumor antigen associated with glioblastoma, ovarian cancer, cervical cancer, head and neck cancer, liver cancer, prostate cancer, pancreatic cancer, renal cell carcinoma, bladder cancer, or a hematologic malignancy.

3. The composition according to claim 1, wherein the guide sequence is specific for the AAVS1 locus.

4. The composition according to claim 2, wherein the gRNA guide sequence comprises SEQ ID NO: 120.

5. The composition according to claim 1, wherein the transgene comprises a sequence encoding an artificial cell death polypeptide.

6. The composition according to claim 1, wherein the guide sequence is specific for the B2M or CIITA locus.

7. The composition according to claim 5, wherein the gRNA guide sequence is specific for the B2M locus and comprises SEQ ID NO: 121.

8. The composition according to claim 5, wherein the gRNA guide sequence is specific for the CIITA locus and comprises SEQ ID NO: 122 or 126.

9. The composition according to claim 1, wherein the transgene comprises a sequence encoding an exogenous cytokine.

10. The composition according to claim 1, wherein the guide sequence is specific for the B2M or CIITA locus.

11. The composition according to claim 9, wherein the gRNA guide sequence is specific for the B2M locus and comprises SEQ ID NO: 121.

12. The composition according to claim 9, wherein the gRNA guide sequence is specific for the CIITA locus and comprises SEQ ID NO: 122 or 126.

13. The composition according to claim 1, wherein the gRNA guide sequence is specific for the NKG2A locus and comprises SEQ ID NO: 124.

14. The composition according to claim 1, wherein the gRNA guide sequence is specific for the TRAC locus and comprises SEQ ID NO: 125.

15. The composition according to claim 1, wherein the gRNA guide sequence is specific for the CLYBL locus and comprises SEQ ID NO: 123.

16. The composition according to claim 1, wherein the gRNA guide sequence is specific for the CD70 locus and comprises SEQ ID NO: 127.

17. The composition according to claim 1, wherein the gRNA guide sequence is specific for the CD38 locus and comprises SEQ ID NO: 128.

18. The composition according to claim 1, wherein the gRNA guide sequence is specific for the CD33 locus and comprises SEQ ID NO: 129 or 130.

19. The composition according to claim 1, wherein the left and right polynucleotide sequences that are homologous to the left and right arms of the target sequence of the AAVS1 comprise the nucleotide sequence of SEQ ID NOs: 60 and 61, respectively, or a fragment thereof.

20. The composition according to claim 1, wherein the left and right polynucleotide sequences that are homologous to the left and right arms of the target sequence of the B2M comprise the nucleotide sequence of SEQ ID NOs: 63 and 64, respectively, or a fragment thereof.

21. The composition according to claim 1, wherein the left and right polynucleotide sequences that are homologous to the left and right arms of the target sequence of the CIITA comprise the nucleotide sequence of (i) SEQ ID NOs: 66 and 67, respectively, or (ii) SEQ ID NOs: 106 and 107, respectively, or a fragment thereof.

22. The composition according to claim 1, wherein the left and right polynucleotide sequences that are homologous to the left and right arms of the target sequence of the CLYBL comprise the nucleotide sequence of SEQ ID NOs: 69 and 70, respectively, or a fragment thereof.

23. The composition according to claim 1, wherein the left and right polynucleotide sequences that are homologous to the left and right arms of the target sequence of the CD70 comprise the nucleotide sequence of SEQ ID NOs: 109 and 110, respectively, or a fragment thereof.

24. The composition according to claim 1, wherein the left and right polynucleotide sequences that are homologous to the left and right arms of the target sequence of the NKG2A comprise the nucleotide sequence of SEQ ID NOs: 72 and 73, respectively, or a fragment thereof.

25. The composition according to claim 1, wherein the left and right polynucleotide sequences that are homologous to the left and right arms of the target sequence of the TRAC comprise the nucleotide sequence of SEQ ID NOs: 75 and 76, respectively, or a fragment thereof.

26. The composition according to claim 1, wherein when the RNP complex is introduced into the cell, expression of an endogenous gene comprising the target sequence complementary to the guide sequence of the gRNA molecule is reduced or eliminated in said cell.

27. The composition according to claim 1, wherein the cell is an induced pluripotent stem cell (iPSC).

28. An iPSC transformed with a transgene by the composition of claim 1.

29.-33. (canceled)

34. An engineered immune-effector cell, or a population thereof, derived from the iPSC of claim 28.

35.-36. (canceled)

37. A MAD7/gRNA ribonucleoprotein (RNP) complex composition for insertion of a transgene, comprising:
(I) a MAD7 nuclease system, wherein the system is encoded by one or more vectors comprising (a) a sequence encoding a guide RNA (gRNA), wherein the sequence is operably linked to a first regulatory element, wherein the gRNA comprises a guide sequence capable of hybridizing to a target sequence of an AAVS1, B2M, CIITA, NKG2A, TRAC, CD70, CD38, CD33, or CLYBL loci in a cell, wherein the guide sequence is selected from SEQ ID NOs: 120-130, and wherein when transcribed, the guide sequence directs sequence-specific binding of the MAD7 complex to the target sequence, and (b) a sequence encoding a MAD7 nuclease, wherein the sequence is operably linked to a second regulatory element; and
(II) a transgene vector comprising: (1) left and right polynucleotide sequences that are homologous to left and right arms of the target sequence of the AAVS1, B2M, CIITA, NKG2A, TRAC, CD70, CD38, CD33, or CLYBL loci, (2) a promoter which is operably linked to (3) a polynucleotide encoding the transgene, and (4) a transcription terminator sequence.

38. A MAD7/gRNA ribonucleoprotein (RNP)-based vector system, comprising:
(I) one or more vectors comprising (a) a sequence encoding a guide RNA (gRNA), wherein the sequence is operably linked to a first regulatory element, wherein the gRNA comprises a guide sequence capable of hybridizing to a target sequence of an AAVS1, B2M, CIITA, NKG2A, TRAC, CD70, CD38, CD33, or CLYBL loci in a cell, wherein the guide sequence is selected from SEQ ID NOs: 120-130, wherein when transcribed, the guide sequence directs sequence-specific binding of the MAD7 complex to the target sequence; (b) a sequence encoding a MAD7 nuclease, wherein the sequence is operably linked to a second regulatory element; and
(II) a transgene vector comprising: (1) left and right polynucleotide sequences that are homologous to left and right arms of the target sequence of the AAVS1, B2M, CIITA, NKG2A, TRAC, CD70, CD38, CD33, or CLYBL loci, (2) a promoter which is operably linked to (3) a polynucleotide encoding the transgene, and (4) a transcription terminator sequence.

39.-63. (canceled)

64. One or more retroviruses comprising the vector system according to claim 38.

65. An iPSC transformed with the vector system according to claim 38.

66.-70. (canceled)

71. An immune-effector cell, or a population thereof, derived from the iPSC of claim 65.

72. A pharmaceutical composition comprising the immuno-effector cell derived from the iPSC of claim 28.

73. A method for preventing or treating a cancer, the method comprising administering, to an individual in need thereof, a pharmaceutically effective amount of the immune-effector cell or the population of any one of claim 34.

74. (canceled)

75. A gRNA comprising a guide sequence selected from the group consisting of SEQ ID NOs: 120-130.

76. An iPSC transformed with the one or more retroviruses according to claim 64.

77. A pharmaceutical composition comprising the immuno-effector cell derived from the iPSC of claim 65.

78. A method for preventing or treating a cancer, the method comprising administering, to an individual in need thereof, a pharmaceutically effective amount of the immune-effector cell or the population of claim 71.

79. A method for preventing or treating a cancer, the method comprising administering, to an individual in need thereof, a pharmaceutically effective amount of the pharmaceutical composition of claim 72.


SEQUENCE LISTING (130 sequences)

SEQ ID NO: 1 - protein, 22 aa, Homo sapiens
MLLLVTSLLLCELPHPAFLLIP

SEQ ID NO: 2 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, Artificial Sequence
  Whitlow Linker
GSTSGSGKPGSGEGSTKG

SEQ ID NO: 4 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, Artificial Sequence
  FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, Artificial Sequence
  (G4S)3
GGGGSGGGGSGGGGS

SEQ ID NO: 26 - protein, 20 aa, Artificial Sequence
  Linker 3
GGSEGKSSGSGSESKSTGGS

SEQ ID NO: 27 - protein, 8 aa, Artificial Sequence
  Linker 4
GGGSGGGS

SEQ ID NO: 28 - protein, 12 aa, Artificial Sequence
  Linker 5
GGGSGGGSGGGS

SEQ ID NO: 29 - protein, 16 aa, Artificial Sequence
  Linker 6
GGGSGGGSGGGSGGGS

SEQ ID NO: 30 - protein, 20 aa, Artificial Sequence
  Linker 7
GGGSGGGSGGGSGGGSGGGS

SEQ ID NO: 31 - protein, 20 aa, Artificial Sequence
  Linker 8
GGGGSGGGGSGGGGSGGGGS

SEQ ID NO: 32 - protein, 25 aa, Artificial Sequence
  Linker 9
GGGGSGGGGSGGGGSGGGGSGGGGS

SEQ ID NO: 33 - protein, 14 aa, Artificial Sequence
  Linker 10
IRPRAIGGSKPRVA

SEQ ID NO: 34 - protein, 15 aa, Artificial Sequence
  Linker 11
GKGGSGKGGSGKGGS

SEQ ID NO: 35 - protein, 15 aa, Artificial Sequence
  Linker 12
GGKGSGGKGSGGKGS

SEQ ID NO: 36 - protein, 15 aa, Artificial Sequence
  Linker 13
GGGKSGGGKSGGGKS

SEQ ID NO: 37 - protein, 15 aa, Artificial Sequence
  Linker 14
GKGKSGKGKSGKGKS

SEQ ID NO: 38 - protein, 15 aa, Artificial Sequence
  Linker 15
GGGKSGGKGSGKGGS

SEQ ID NO: 39 - protein, 15 aa, Artificial Sequence
  Linker 16
GKPGSGKPGSGKPGS

SEQ ID NO: 40 - protein, 20 aa, Artificial Sequence
  Linker 17
GKPGSGKPGSGKPGSGKPGS

SEQ ID NO: 41 - protein, 20 aa, Artificial Sequence
  Linker 18
GKGKSGKGKSGKGKSGKGKS

SEQ ID NO: 42 - protein, 14 aa, Artificial Sequence
  Linker 19
STAGDTHLGGEDFD

SEQ ID NO: 43 - protein, 15 aa, Artificial Sequence
  Linker 20
GEGGSGEGGSGEGGS

SEQ ID NO: 44 - protein, 15 aa, Artificial Sequence
  Linker 21
GGEGSGGEGSGGEGS

SEQ ID NO: 45 - protein, 15 aa, Artificial Sequence
  Linker 22
GEGESGEGESGEGES

SEQ ID NO: 46 - protein, 15 aa, Artificial Sequence
  Linker 23
GGGESGGEGSGEGGS

SEQ ID NO: 47 - protein, 20 aa, Artificial Sequence
  Linker 24
GEGESGEGESGEGESGEGES

SEQ ID NO: 48 - protein, 19 aa, Artificial Sequence
  Linker 26
PRGASKSGSASQTGSAPGS

SEQ ID NO: 49 - protein, 19 aa, Artificial Sequence
  Linker 27
GTAAAGAGAAGGAAAGAAG

SEQ ID NO: 50 - protein, 19 aa, Artificial Sequence
  Linker 28
GTSGSSGSGSGGSGSGGGG

SEQ ID NO: 51 - protein, 4 aa, Artificial Sequence
  Linker 30
GSGS

SEQ ID NO: 52 - protein, 10 aa, Artificial Sequence
  Linker 31
APAPAPAPAP

SEQ ID NO: 53 - protein, 20 aa, Artificial Sequence
  Linker 32
APAPAPAPAPAPAPAPAPAP

SEQ ID NO: 54 - protein, 32 aa, Artificial Sequence
  Linker 33
AEAAAKEAAAKEAAAAKEAAAAKEAAAAKAAA

SEQ ID NO: 55 - protein, 15 aa, Artificial Sequence
  Hinge
EPKSCDKTHTCPPCP

SEQ ID NO: 56 - protein, 12 aa, Artificial Sequence
  Hinge
ERKCCVECPPCP

SEQ ID NO: 57 - protein, 62 aa, Artificial Sequence
  Hinge
ELKTPLGDTTHTCPRCPEPKSCDTPPPCPRCPEPKSCDTPPPCPRCPEPKSCDTPPPCPRCP

SEQ ID NO: 58 - protein, 12 aa, Artificial Sequence
  Hinge
ESKYGPPCPSCP

SEQ ID NO: 59 - DNA, 24 bp, Artificial Sequence
  Targeting domain
tttatctgtcccctccaccccaca

SEQ ID NO: 60 - DNA, 1205 bp, Artificial Sequence
  AAVS1 Left Homology Arm
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 61 - DNA, 1200 bp, Artificial Sequence
  AAVS1 Right Homology Arm
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 62 - DNA, 24 bp, Artificial Sequence
  Targeting domain
tttactcacgtcatccagcagaga

SEQ ID NO: 63 - DNA, 1042 bp, Artificial Sequence
  B2M Left Homology Arm
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 64 - DNA, 1023 bp, Artificial Sequence
  B2M Right Homology Arm
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 65 - DNA, 24 bp, Artificial Sequence
  Targeting domain
tttaccttggggctctgacaggta

SEQ ID NO: 66 - DNA, 1000 bp, Artificial Sequence
  CIITA Left Homology Arm
acaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcag

SEQ ID NO: 67 - DNA, 1000 bp, Artificial Sequence
  CIITA Right Homology Arm
agccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaaggga

SEQ ID NO: 68 - DNA, 24 bp, Artificial Sequence
  Targeting domain
agagtgatcacagctctgactaaa

SEQ ID NO: 69 - DNA, 1046 bp, Artificial Sequence
  CLYBL Left Homology Arm
gtccagactcaggaggacttagttctcttcataaaaagttgatcacctttgctttctcctttgcatgggcaataaagaagtgaaaaataaattcccagtgagcttgcattctgcttgggaacaacatagcacatccattttctagagagctgtccagtccccatttgagggctgctagccacatgtcaagcaggaacccagagtataaccaggaaaatgtctgtggtagaccccagtgattcatgcctcccatctccacaccctcctgtagtcccctcccacgctgattctggactaggccacatggcttggccagtggaacacatgcaggcttgcacgtctggaactctgccacctaatttaggatcccagactctcctactggagacacaggtccttagtgacagtctgcaccaccattcagacaagtcagtagggccatcttagatcatccagccctagtcaagccaccagataactgtacccacataagtgacccctggcgagaccagcaggagaatcatgccaatgggccaatatacattctgacccacagtttcataataaaataaaatggttgtggttgtaagccactatgtttcagagtggtttgttacacagcaataaataactaatatagtaggcataccatcaagtccaaagtaggtagagaagaatgtaaatagcagagcaaaacagcatgactggtggctgggaggcttaaaactgggacaggatcagagtcatgaaagaagtcaaagaaatggttcagaagtaaggctgagactgacttacaaaagctgaaagtccctttaagttggtgtttggtgcattggcaggggcaggtatggtgacttaaaagagccatgctcaacaagatcaagcacaacacaatcacgggtcaccccagcagaccttagcgagtctagccatttctttggtggtggtcacagtcatgcttcagcccagtttccacttggacaaatggtacatattttcaatgagatgaaaattaagatacaatccatgtgctcagagagtgatca

SEQ ID NO: 70 - DNA, 1053 bp, Artificial Sequence
  CLYBL Right Homology Arm
atcatgtctgctcgacagctctgactaaacactgtgccccaaagtgttgaggaattgggaaaacctagctgagttagtggtctcttttctgttacaataaagctcataatgaaaattagccttctttgttcttccccaagtctttctttctagacgaaactactttcaactgttttaacttccttactgttaacttccatatttctagatagtatggtcagactgttatttcttgacttttaaatgtaagatattatctactgacttccttctatgtaagatgaggattgagctctcttacccttctcccatttcctcatccttccaacataaatatattttgggattatatcaacattcaatgttacttaaagtgaccttgtaaatattttcacaactgagccatgtttgatttgtatacttatgtttactttactgtttttcctgaagttaataattgccttgaatttatttatttctttaaaaatgtttcattactcaggactgtagtttacattacgattctttgtgttatacagttgatgggtttcttttctttcttaatttctttaaaaaatagagatggggtcttactatattacccaggctggtcttgaagtcctgggctcaagtgatcttcctgtctcagcctaccaagtagctgagactataggtgcaaaaaagccactatacctggctagtttacaggttttaacaaatgcattatgccacgtatccattattacaggatcacacaagatattttcattaccctgaaagcatccctgtgttccaccaattcatcctgcctccatgagccgctggcaaccactgatctctatagttttgccttttctaaaatgtcatataattggaatcatacagtctgtagcattttcagactagcttttaaaatttggcaatatgcatttaaggttcctccttaaatgtgaagggcatagccaatgtggcttgatagctcatttctttttattggtgaatatttcattgtctggatgttccacagtttgtttatcctcgagagcttggcgt

SEQ ID NO: 71 - DNA, 24 bp, Artificial Sequence
  Targeting domain
ctcagacctgaatctgcccccaaa

SEQ ID NO: 72 - DNA, 1045 bp, Artificial Sequence
  NKG2A Left Homology Arm
gacctggcctccgttttatgtagtgttcctaatactatttaggttatgcataagttaactgtgtcacatagcattttctgtcatatacaaatatttaattttttataatttttaataaataatttttaataatttcttagaacatttcagccattaagaaaagaccatttaattacctctattgattttcattaatgtgtataaattactaattaaaagtaatgtagtgcaagaaatttttatatatatatatgtcaactttactattactcatgtgcagaccacatagtcttaaccattactggatttcaaaattaacataaaaatgagtgcagaagtttttctaatacaacattttaatttttaacacaggtaaaacatcagactttaagaaatatatttttattcctgaacttctttattccttagtaatttattgcttctcattgccccagcaataatattttgtcaaatgcagaaaatttatcttttttttttgagtcggagtctcactctgtcgcccaggctggagtgcagtggcacaatctccgctcactgcaacctctgccgcccatgttcaagagattctcctgcctcagcctcccgagtagctaggactacaggcgcctgacatcatgcccgactactttttgtatttttagtagagacggagtttcaccgtgttagccaggatggtctcgatctcctgacctcgtgatcggcatgcctcggcctcccaaagtgctgggattacaggcgtgagccaccgcgcccggcctaaaaatctttttttaaaacaaatattcataagaaacgtgtttaggcttgaagaaaatcagagaaagaactttagattatttaatgcaaaatgagctccaatactcgttctccacctcacccttttaattgcactagggaatcctgtatataaaccatttattaacttcttaactactgttattatagagtacagtccctgacatcacacactgcagagatggataaccaaggagtaatctactcagacctgaacggtaccaacgcg

SEQ ID NO: 73 - DNA, 1020 bp, Artificial Sequence
  NKG2A Right Homology Arm
aatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaaggtaaagcatttaaaagatcctcaatataacagtctaggatgtgcagcttggggtacaggaatgtggggaaagagaagggagtgctcatatatcttctatttgcaaagatcagaattccaagttgagatatgctatttcaatgtaaagtatgaagactgattgaactcattgttgaagtttgtagtctttgtcaaataattcatggagcattatttttcctgaaaattcaatggtatattattctgagaaaaagattacaatgggagatgagggtttggggtccaagtttctctgtatgattcctgtgcattcaggttctcttgtctgtgaatcttctaaacgactgtatccacctctcctttcgcactgttcccatttctctccctgcagatttaccatcagctccagagaagctcattgttgggatcctgggaattatctgtcttatcttaatggcctctgtggtaacgatagttgttattccctgtaagtctattttcgaagattacaaggggaattttcacgttaatgattgaatgtgcctctaaacatttcatattttcagggaatagagttctcattgtaatgtatatatttggactaaatgtggaatgattattctgaatttgtcaaagaataaatgaaagaataattgttgaaagtattcgcttctgatgcaatcgtatgtatatatttggatttcataactcaaaaatatgttctaggagtctgaaaaaccttactgagaaatagaaattaatttttgaaagtagttaaatcaagaattataagaactatatgagatggtgaaatttggttctttagatctatgaaatacttttccaaaaaaccaccattactttatc

SEQ ID NO: 74 - DNA, 24 bp, Artificial Sequence
  Targeting domain
gtgtaccagctgagagactctaaa

SEQ ID NO: 75 - DNA, 1229 bp, Artificial Sequence
  TRAC Left Homology Arm
cgactctagactcgaagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctcgagaaggatctg

SEQ ID NO: 76 - DNA, 1130 bp, Artificial Sequence
  TRAC Right Homology Arm
atcatgtctgggatctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaattgatccgatccgagct

SEQ ID NO: 77 - protein, 371 aa, Artificial Sequence
  tEGFR
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 78 - protein, 19 aa, Artificial Sequence
  P2A
ATNFSLLKQAGDVEENPGP

SEQ ID NO: 79 - protein, 162 aa, Artificial Sequence
  IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 19 aa, Artificial Sequence
  Signal Sequence
MEFGLSWVFLVALFRGVQC

SEQ ID NO: 81 - protein, 21 aa, Artificial Sequence
  CD79b Epitope
ARSEDRYRNPKGSACSRIWQS

SEQ ID NO: 82 - protein, 10 aa, Artificial Sequence
  CD20 Mimitope
ACPYANPSLC

SEQ ID NO: 83 - protein, 8 aa, Artificial Sequence
  Linker
GGGSGGGS

SEQ ID NO: 84 - protein, 176 aa, Artificial Sequence
  ErbB Epitope
EGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGG

SEQ ID NO: 85 - protein, 153 aa, Artificial Sequence
  IL-2 Amino Acid Sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 556 aa, Artificial Sequence
  tEGFR-P2A-IL15 Amino Acid Sequence
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 87 - protein, 5 aa, Artificial Sequence
  Linker
GGGGS

SEQ ID NO: 88 - protein, 287 aa, Artificial Sequence
  CD79b-P2A-IL15
MEFGLSWVFLVALFRGVQCARSEDRYRNPKGSACSRIWQSTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 89 - protein, 296 aa, Artificial Sequence
  CD20 Mimitope-P2A-IL15
MEFGLSWVFLVALFRGVQCACPYANPSLCGGGGSGGGGSACPYANPSLCTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 90 - protein, 376 aa, Artificial Sequence
  ErbB Epitope-P2A-IL15
MEFGLSWVFLVALFRGVQCEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 91 - protein, 335 aa, Artificial Sequence
  HLA-E
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 92 - protein, 33 aa, Artificial Sequence
  HLA-G Signal Peptide
MVVMAPRTLFLLLSGALTLTETWAVMAPRTLIL

SEQ ID NO: 93 - protein, 504 aa, Artificial Sequence
  HLA-G Signal Peptide-B2M-HLA-E Amino Acid
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 94 - DNA, 1515 bp, Artificial Sequence
  HLA-G Signal Peptide-B2M-HLA-E Nucleotide
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 95 - protein, 312 aa, Artificial Sequence
  HLA-G
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 96 - protein, 471 aa, Artificial Sequence
  HLA-G Signal Peptide-B2M-HLA-G
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 97 - DNA, 1422 bp, Artificial Sequence
  HLA-G Signal Peptide-B2M-HLA-G
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 98 - DNA, 1724 bp, Artificial Sequence
  CAG Promoter
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 99 - DNA, 121 bp, Artificial Sequence
  SV40 Terminator
aacttgtttattgcagcttataatggttacaaataaagcaatagcatcacaaatttcacaaataaagcatttttttcactgcattctagttgtggtttgtccaaactcatcaatgtatctt

SEQ ID NO: 100 - DNA, 1668 bp, Artificial Sequence
  tEGFR-P2A-IL15 Nucleotide Seqeuence
atgaggccctcaggcactgccggggccgccctcctggccctgttagccgctttgtgtccagcaagccgcgccggagtgcggaaatgtaagaaatgcgaaggaccctgccggaaggtatgcaacggcattgggattggcgaattcaaggacagcctgagcattaatgctacaaacatcaagcactttaagaattgcaccagcattagcggcgatctgcatatactgccagtggctttccgaggcgactcttttactcatacccctccgctggaccctcaagagctggacattctcaagactgtgaaggaaattacggggtttctgctcattcaggcctggcctgaaaaccgcacggatttgcatgcctttgagaatctggaaataatcagaggccggacgaaacagcatggccagttcagcctcgcggtcgtctctttgaatattacgtcactcggcctcaggtccctcaaagagatttctgatggcgatgtcatcatctctggtaataagaatctgtgttacgcaaataccatcaattggaagaagctctttgggacctcaggtcaaaagactaaaattatctccaaccgcggcgagaacagctgtaaggctacaggccaggtttgccacgcgctctgctccccagagggttgctgggggcctgagccaagggattgcgtttcatgtcgcaacgtgtctcggggcagagaatgcgtggataaatgtaacctcttagagggcgaacctcgcgagtttgttgagaactcagaatgtatacagtgccaccccgaatgtcttcctcaggccatgaatatcacatgcaccggacgcggaccagacaactgtatccaatgtgctcactacattgacggacctcattgtgtgaaaacatgccccgcaggagttatgggagaaaacaacaccctcgtttggaaatatgccgatgcaggtcacgtatgtcacctgtgccacccaaactgcacttatgggtgcaccgggccgggcctggaggggtgccctacgaatggaccaaaaattcccagtattgcaactgggatggtcggggcactgttgttgctgcttgtggttgccctcgggataggcctgtttatgtctggctccggcgccaccaatttcagcctgctgaaacaggcaggcgacgtcgaagaaaatccaggaccaatgcgaatatcaaaaccacacttgcgcagcatttctatacagtgctatttgtgcttgttgctgaactctcacttcctcacagaggctgggatacacgttttcatacttggatgtttttcagctgggctgccgaagacagaggcgaattgggtgaatgtaatttcagacctcaagaagatcgaggatctcatccagtccatgcacatcgacgctactctgtacacagagagcgatgtccacccttcttgtaaggttaccgccatgaaatgcttccttttggaactccaagtcatctcattggaatcaggggatgcgtccattcatgacaccgtggaaaacctgataatactggctaacaacagcttgtcaagtaatgggaatgttactgagtccggttgtaaagaatgtgaagagctggaggagaagaacattaaggaatttttgcaatcttttgtacatattgttcagatgtttattaacacaagc

SEQ ID NO: 101 - protein, 10 aa, Artificial Sequence
  (G4S)2
GGGGSGGGGS

SEQ ID NO: 102 - protein, 30 aa, Artificial Sequence
  (G4S)6
GGGGSGGGGSGGGGSGGGGSGGGGSGGGGS

SEQ ID NO: 103 - protein, 35 aa, Artificial Sequence
  (G4S)7
GGGGSGGGGSGGGGSGGGGSGGGGSGGGGSGGGGS

SEQ ID NO: 104 - protein, 40 aa, Artificial Sequence
  (G4S)8
GGGGSGGGGSGGGGSGGGGSGGGGSGGGGSGGGGSGGGGS

SEQ ID NO: 105 - DNA, 24 bp, Artificial Sequence
  Targeting domain
tttctgcccaacttctgctggcat

SEQ ID NO: 106 - DNA, 1050 bp, Artificial Sequence
  CIITA Ex5 Left Homology Arm Sequence
aaacttctaccaccgtcacctatctctcagggtttcctaaacatactctgaacaagtttcctcactctgccactgtgacccagaaagctatctgttctccttctcccagactctgcctcatctttcaaggcctggttccaggatcccttcctccaggaagccttccctgattgccctattccaccatacaccttttttctcggacttcatgtcatggtgcctatattccaagggctctgagccatgtacccctttatatagttaccactatttactgagtgcctactgtataccagctactgtgttggatgctctagatgtagaacctctaattatcaccatcgattcctgggtagtaggcattatttattcatcttacacagatgagaaaatggaggcccacagtggttaaataagtagcccaagattgcacagctagtagggctagtggaaagtagaggtggaatttgaactcaaatcctgcagtaactctaccattctgccttgctcttctttgtagcagtagataagttttcatggatacatacctcatccttttgattagattaagggcccctggagtgtcagtgttcattcatttgtttgatcattcattcattcaacaaacatttcttgagtccccactgtgtgccaggcccagaggttccccagcccaaggcctggcacacagtgggccttcagttagaccttgttgattgactgcgcttttccttgtctgggcagcggaactggaccagtatgtcttccaggactcccagctggagggcctgagcaaggacattttcagtaagtttgtggtgggtggggaggtcttggctcagcctgcatttcctgccttgttccctggggggtgccctaatacctgacgaccattcattgatgggcagtcagacccctctccccaaggtgggtacaatagagactcaccttgggctttcattgattgtgtgagttggtctctggtttttctcaaagtagagcacataggaccagatgaagtgatcggtgagagtatggagatgccagcag

SEQ ID NO: 107 - DNA, 1052 bp, Artificial Sequence
  CIITA Ex5 Right Homology Arm Sequence
aagttgggcagaaaagtcagaaaagacgtgagtgagcccctccctgatccaacctagccttgcttgagacctggcctttccttgactccaaagcctgctgtgggtccaacttgcttccctcgctaagtcctgtctggttgggaggccctttaaaagccaacaggagccttaaaatgtacatctgattatttcatggccctgataaccctccaatggctacaaaatacatgccacaaggcctgtatggcccttcctccctctccaaacccactatgagacaccacacttcagccaccaccagcttctccactcctataacccatgtgccctttcaagcctcggggcctttgcagttgctatagtctctatctggaatgcccttcccccagttcttcccatggctgactcctttgaatctttctggtgttggctaaactgtcacctcttcctggaacccttctctgaccatccttccatgtagattagctcagttattctcaccttgtgtgtctttttccttgcagtttagcactcattaccatctggacatattttacgccttgctctcccactgtgaggacagggaccttgtctttcttgctcgtgactgtttccccagcatctagtgcagtgcctggtatgcagtagcacctcagtagatatctgttgaatgaaaacatctgtaaaatgggtgtaacagttaactgagtacttattatgggtctgaccatgtgtaagtcctgtatctatttattcagttcttaaacaggtgaatcgcacacagggtatgagattttaaaagtgcaaagaatattcagtgaaggctgggcgcagcggctcacacctgtaatcccagcagtttgggaggccaagggggacggatcacttgaggtcaggagtttgatacctgcctggccaacatggtgaaaccgcgtctctaccaaaaaatacaaaaattagccgggtgtggtggtgcacgcctgtaatcccagctactcgggaggctgaggcaggagaatcgcttgaacccaggaggtggaggttgcagtgag

SEQ ID NO: 108 - DNA, 24 bp, Artificial Sequence
  Targeting domain
tttggtcccattggtcgcgggctt

SEQ ID NO: 109 - DNA, 973 bp, Artificial Sequence
  CD70 Ex1 Left Homology Arm Sequence
aaaaataaaaaataaaaatattaacttaatttaactttaaacaaaaaagcaggtggtctccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggt

SEQ ID NO: 110 - DNA, 1000 bp, Artificial Sequence
  CD70 Ex1 Right Homology Arm Sequence
cgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagagg

SEQ ID NO: 111 - DNA, 23 bp, Artificial Sequence
  Targeting domain
tttcccgagaccgtcctggcgcg

SEQ ID NO: 112 - DNA, 25 bp, Artificial Sequence
  Targeting domain
tttgtctgcagggaaacaagagacc

SEQ ID NO: 113 - DNA, 25 bp, Artificial Sequence
  Targeting domain
tttggagtggccgggttctagagtg

SEQ ID NO: 114 - DNA, 3792 bp, Artificial Sequence
  WT MAD7 nucleic acid sequence
atgaataatggaacaaataactttcagaattttatcggaatttcttctttgcagaagactcttaggaatgctctcattccaaccgaaacaacacagcaatttattgttaaaaacggaataattaaagaagatgagctaagaggagaaaatcgtcagatacttaaagatatcatggatgattattacagaggtttcatttcagaaactttatcgtcaattgatgatattgactggacttctttatttgagaaaatggaaattcagttaaaaaatggagataacaaagacactcttataaaagaacagactgaataccgtaaggcaattcataaaaaatttgcaaatgatgatagatttaaaaatatgttcagtgcaaaattaatctcagatattcttcctgaatttgtcattcataacaataattattctgcatcagaaaaggaagaaaaaacacaggtaattaaattattttccagatttgcaacgtcattcaaggactattttaaaaacagggctaattgtttttcggctgatgatatatcttcatcttcttgtcatagaatagttaatgataatgcagagatattttttagtaatgcattggtgtataggagaattgtaaaaagtctttcaaatgatgatataaataaaatatccggagatatgaaggattcattaaaggaaatgtctctggaagaaatttattcttatgaaaaatatggggaatttattacacaggaaggtatatctttttataatgatatatgtggtaaagtaaattcatttatgaatttatattgccagaaaaataaagaaaacaaaaatctctataagctgcaaaagcttcataaacagatactgtgcatagcagatacttcttatgaggtgccgtataaatttgaatcagatgaagaggtttatcaatcagtgaatggatttttggacaatattagttcgaaacatatcgttgaaagattgcgtaagattggagacaactataacggctacaatcttgataagatttatattgttagtaaattctatgaatcagtttcacaaaagacatatagagattgggaaacaataaatactgcattagaaattcattacaacaatatattacccggaaatggtaaatctaaagctgacaaggtaaaaaaagcggtaaagaatgatctgcaaaaaagcattactgaaatcaatgagcttgttagcaattataaattatgttcggatgataatattaaagctgagacatatatacatgaaatatcacatattttgaataattttgaagcacaggagcttaagtataatcctgaaattcatctggtggaaagtgaattgaaagcatctgaattaaaaaatgttctcgatgtaataatgaatgcttttcattggtgttcggttttcatgacagaggagctggtagataaagataataatttttatgccgagttagaagagatatatgacgaaatatatccggtaatttcattgtataatcttgtgcgtaattatgtaacgcagaagccatatagtacaaaaaaaattaaattgaattttggtattcctacactagcggatggatggagtaaaagtaaagaatatagtaataatgcaattattctcatgcgtgataatttgtactatttaggaatatttaatgcaaaaaataagcctgacaaaaagataattgaaggtaatacatcagaaaataaaggggattataagaagatgatttataatcttctgccaggaccaaataaaatgatccccaaggtattcctctcttcaaaaaccggagtggaaacatataagccgtctgcctatatattggagggctataaacaaaacaagcatattaaatcctctaaggattttgatataacattttgtcacgatttgattgattattttaagaactgtatagcaatacatcctgaatggaagaattttggctttgatttttctgacacctccacatatgaagatatcagcggattttacagagaagtcgaattacaaggttataaaatcgactggacatatatcagcgaaaaggatattgatttgttgcaggaaaaaggacagttatatttattccaaatatataacaaagatttttccaagaaaagtaccggaaatgataatcttcatactatgtatttgaagaatttgtttagtgaagagaatttaaaggatattgtactgaaattaaacggtgaggcggaaatcttctttagaaaatcaagcataaagaatccaataattcataaaaaaggctctattcttgttaatagaacatatgaagcagaggaaaaagatcaatttggaaatatccagatagtcagaaaaaacataccggaaaatatatatcaggagctttataaatatttcaatgataaaagtgataaagaactttcggatgaagcagctaagcttaagaatgtagtaggtcatcatgaggctgctacaaacatagtaaaagattatagatatacatatgataaatattttcttcatatgcctattacaatcaattttaaagccaataagacaggctttattaatgacagaatattacaatatattgctaaagaaaaggatttgcatgtaataggcattgatcgtggtgaaagaaacctgatatatgtttcagtaattgatacttgtggaaatattgttgaacaaaaatcgtttaacattgttaatggatatgattatcagattaagctcaagcagcaggagggggcgcgacaaatcgcacgaaaagaatggaaagaaatcggcaaaataaaagaaattaaagaaggctatttatctcttgtaattcatgaaatttcaaagatggttattaaatataatgccataattgcaatggaggatttaagctacggatttaaaaaaggtcgtttcaaggttgagcgacaggtttaccagaagtttgagacaatgcttatcaacaaactcaactatctggtatttaaagatatatccataacggaaaacggtggtcttctaaagggataccagcttacatatattccagataaactgaaaaatgtgggtcatcaatgtggctgtatattttatgtacctgctgcctatacatcaaaaatagatcctacaaccggatttgtaaatatattcaaatttaaagatttaacagttgatgcgaagagagaatttataaaaaaatttgacagtatcagatatgattcagaaaaaaatctgttttgttttacattcgattataataactttattacgcaaaatactgttatgtcaaagtcaagctggagtgtatatacgtacggagttaggataaaaagaagatttgtcaatggcaggttctcaaatgaatcggatacaattgatataacaaaagatatggaaaaaacactcgaaatgacagatataaattggagagatggtcatgatctgaggcaggatattattgattatgaaatcgtacaacacatatttgagatttttagattgactgtacaaatgagaaacagtttaagtgaattagaagacagggattatgaccgtttgatttctccggtgctcaatgaaaataatatattttatgattcagctaaagcaggagatgcgttacctaaagacgcagatgctaatggtgcatattgtatagctctaaaaggcttgtatgaaatcaaacaaattacagagaattggaaagaagacggtaagttttcaagagataaacttaaaatttccaataaggactggtttgactttattcaaaataaaaggtatttataa

SEQ ID NO: 115 - DNA, 3792 bp, Artificial Sequence
  Codon optimized nucleic acid sequence
atgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 116 - protein, 1263 aa, Artificial Sequence
  MAD7 amino acid sequence
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 117 - DNA, 35 bp, Artificial Sequence
  Scaffold sequence
gttaagttatatagaataatttctactgttgtaga

SEQ ID NO: 118 - DNA, 36 bp, Artificial Sequence
  Scaffold sequence
ctctacaactgataaagaatttctacttttgtagat

SEQ ID NO: 119 - DNA, 36 bp, Artificial Sequence
  Scaffold sequence
gtctggccccaaattttaatttctactgttgtagat

SEQ ID NO: 120 - RNA, 24 nt, Artificial Sequence
  Guide RNA
uuuaucuguccccuccaccccaca

SEQ ID NO: 121 - RNA, 24 nt, Artificial Sequence
  Guide RNA
uuuacucacgucauccagcagaga

SEQ ID NO: 122 - RNA, 24 nt, Artificial Sequence
  Guide RNA
uuuaccuuggggcucugacaggua

SEQ ID NO: 123 - RNA, 24 nt, Artificial Sequence
  Guide RNA
agagugaucacagcucugacuaaa

SEQ ID NO: 124 - RNA, 24 nt, Artificial Sequence
  Guide RNA
cucagaccugaaucugcccccaaa

SEQ ID NO: 125 - RNA, 24 nt, Artificial Sequence
  Guide RNA
guguaccagcugagagacucuaaa

SEQ ID NO: 126 - RNA, 24 nt, Artificial Sequence
  Guide RNA
uuucugcccaacuucugcuggcau

SEQ ID NO: 127 - RNA, 24 nt, Artificial Sequence
  Guide RNA
uuuggucccauuggucgcgggcuu

SEQ ID NO: 128 - RNA, 23 nt, Artificial Sequence
  Guide RNA
uuucccgagaccguccuggcgcg

SEQ ID NO: 129 - RNA, 25 nt, Artificial Sequence
  Guide RNA
uuugucugcagggaaacaagagacc

SEQ ID NO: 130 - RNA, 25 nt, Artificial Sequence
  Guide RNA
uuuggaguggccggguucuagagug